(19) United States
(12) Patent Application Publication
(10) Pub. No.: US 2026/0027535 A1
(43) Pub. Date: Jan. 29, 2026

(54) PROCESS FOR CONTINUOUS CATALYTIC HYDROGENATION OF MDA

(71) Applicant: Evonik Operations GmbH, Essen (DE)

(72) Inventors: Hanns Kuhlmann, Sprockhoevel (DE); Armin Matthias Rix, Marl (DE); Niklas Paul, Marl (DE); Lea Wessner, Dortmund (DE); Maria Vargas Gómez, Haltern am See (DE); Tobias Winkler, Duelmen (DE); Florian Boeck, Witten (DE); Daniel Sudhoff, Bergkamen (DE)

(73) Assignee: Evonik Operations GmbH, Essen (DE)

(21) Appl. No.: 19/276,213

(22) Filed: Jul. 22, 2025

(30) Foreign Application Priority Data
Jul. 26, 2024 (EP) 24191098.3

(51) Int. Cl. B01J 8/02; B01J 4/00; B01J 8/04; C07C 209/72

(52) U.S. Cl. CPC B01J 8/0285; B01J 4/001; B01J 8/0278; B01J 8/0492; B01J 8/0496; C07C 209/72; B01J 2204/002; B01J 2204/007; B01J 2208/00265; C07C 2601/14

(57) ABSTRACT

A plant for hydrogenation of methylenedianiline (MDA; reactant1) with a hydrogen donor (reactant2), especially a gaseous hydrogen donor, preferably hydrogen (H2), including a conditioning unit for the reactants, a reactor unit and a separation unit, wherein the conditioning unit includes at least part of the length of the (feed) conduits for reactant1, reactant2 and at least one solvent, at least one heat exchanger in at least one (feed) conduit, at least one mixer for mixing the reactants and/or at least one reactant with at least one solvent; the reactor unit includes at least one fixed bed reactor as main reactor with an immobile catalyst packing.

### CROSS-REFERENCE TO RELATED APPLICATIONS

This patent application claims priority to European Patent Application No. EP 24191098.3, filed on Jul. 26, 2024, in the European Patent Office, the entire disclosure of which is hereby incorporated by reference herein.

### BACKGROUND OF THE INVENTION

### Field of the Invention

The invention relates to a plant and to a process for continuous catalytic hydrogenation of MDA, especially for production of methylenebis(cyclohexylamine), such as 4,4′-diaminodicyclohexylmethane (PACM) in particular.

Description of Related Art Processes for hydrogenation of organic compounds, especially for hydrogenation of aromatic compounds to the corresponding cyclohexane derivatives, are already known from the related art.

Methylenebis(cyclohexylamine) is a cycloaliphatic amine that is solid or liquid under standard conditions (SATP) and is typically produced by liquid phase hydrogenation of MDA. The acronym MDA was introduced historically as an abbreviation for the product mixture which is formed in the reaction of aniline and formaldehyde and comprises mainly “methylenedianiline” (diaminodiphenylmethane), and is still used to describe the process product, which is now produced on an industrial scale. The hydrogenation product, comprising mainly methylenebis(cyclohexylamine), is therefore often also referred to as H12MDA.

Because of the process for production thereof, MDA is typically a mixture of different diaminodiphenylmethanes. It consists mainly of 4,4′-diaminodiphenylmethane. However, 2,4′ and 2,2′ isomers may also be present. MDA may also contain reaction products having three or more aromatic rings that are formed in the reaction of aniline and formaldehyde, in particular those having three or more phenyl rings. These reaction products having three or more aromatic rings are also referred to as polynuclear compounds.

Because of the high proportion of 4,4′-diaminodiphenylmethane in the MDA used, commercially available methylenebis(cyclohexylamine) is mostly 4,4′-diaminodicyclohexylmethane or bis(para-aminocyclohexyl)methane. Because of the potential presence of the corresponding 2,4′- and 2,2′-diaminophenylmethane isomers in the MDA, 2,4′-diaminodicyclohexylmethane and 2,2′-diaminodicyclohexylmethane may also be present in methylenebis(cyclohexylamine). In addition, hydrogenated MDA may also contain (optionally partly) hydrogenated polynuclear compounds as well as methylenebis(cyclohexylamine).

U.S. Pat. No. 5,578,546 A discloses that a process for producing methylenebis(cyclohexylamine) was first described in 1947 and converted to industrial scale in 1965. Hydrogenation of MDA is highly exothermic. For example, WO 2010/069484 A1 indicates an enthalpy of reaction of −1600 KJ/mol.

As a result of hydrogenation, various diastereoisomers are formed depending on the process. The 4,4′-diaminodicyclohexylmethane (PACM) product derived from 4,4′-diaminodiphenylmethane may take the form of trans/trans, cis/cis and cis/trans isomers and is therefore generally a mixture of these isomers with different proportions. The melting point of the compound rises with rising trans/trans content. Therefore, the fields of application vary significantly depending on the isomer content: While methylenebis(cyclohexylamine) qualities with a low trans/trans content (e.g. 10-30% by weight) are used in the field of amine and isocyanate crosslinkers, especially in the field of two-component resins, qualities with a high trans/trans content (e.g. ≥48% by weight) are mainly used as regulator in polyamide compounds. The production of products with a low trans/trans content in particular is a challenge, since the thermodynamic equilibrium, as described in U.S. Pat. No. 3,636,108 A, is in the range of significantly higher trans/trans contents (up to 51.2%). In addition, U.S. Pat. No. 2,606,925 A shows that the equilibrium can be shifted retrospectively by prolonged heat treatment in the direction of a higher proportion of trans/trans isomer.

The composition of the hydrogenation product also depends on the composition of the MDA used: MDA is often used in qualities from MDA50 to MDA100, with the number between 50 and 100 indicating the content of diaminodiphenylmethanes in the MDA mixture. MDA50 is an MDA quality which, as explained above, for process-related reasons, contains about 50% by weight of diaminodiphenylmethanes and 50% by weight of polynuclear compounds. The individual polynuclear compounds can be referred to as trinuclear compounds, tetranuclear compounds, etc. according to the number of aromatic nuclei present. MDA50 is the quality produced in the highest volume and is mainly processed to methylene dicyclohexyl diisocyanate (MDI). MDA100 is pure

MDA or diaminodiphenylmethane without polynuclear compounds. MDA85 or MDA90 are other grades of medium purity available on the market. When patent specifications relating to the process for production of methylenebis(cyclohexylamine) address the purity of the MDA quality, the quality in question is usually MDA100 (e.g. CN 110204447 B). US 2005/261525 A1, by contrast, prefers the hydrogenation of MDA50. The hydrogenated oligomeric amines obtained here as high boilers are suitable as crosslinkers having particularly low vapour pressures for a range of specialty applications, as shown by US 2004/162409 A1.

The content of (possibly partly) hydrogenated polynuclear compounds in the product decreases in the order of the reactants MDA50, MDA85, MDA90, MDA100, since the content of polynuclear compounds decreases from MDA50 to MDA100.

WO 2009/153123 A1 discloses a continuous process and a reactor for hydrogenation of organic compounds in a polyphasic multistage system in the presence of a homogeneous or heterogeneous catalyst. Catalysts proposed include precious metals such as platinum, palladium, ruthenium and rhodium, or other transition metals such as molybdenum, tungsten and chromium. It is possible here for the heterogeneous catalysts to be disposed on support materials, for example carbon, aluminium oxide, silicon dioxide, zirconium dioxide, zeolites, aluminosilicates or mixtures of these support materials. Substrates used in this process are preferably aromatic compounds containing amino substituents, for example MDA, polymer MDA, aniline, 2,4-diaminotoluene, 2,6-diaminotoluene, o-phenylenediamine, etc. The heterogeneous catalysts are used in suspension.

DE 19533718 A1 discloses a process for hydrogenation of aromatic compounds in which at least one amino group is bonded to an aromatic nucleus. For this purpose, it is possible to use a heterogeneous catalyst containing ruthenium and optionally at least one metal of transition group I, VII or VIII. The support material used is, for example, aluminium oxide, silicon oxide, titanium oxide or zirconium oxide, preferably aluminium oxide or zirconium oxide. Only a catalyst containing ruthenium on the support material aluminium oxide is given as an example, and not zirconium oxide.

EP 1337331 A1 discloses a process for catalytic hydrogenation of aromatic or heteroaromatic amines, wherein ruthenium acts as the active metal and the catalyst contains at least one additional metal of transition group I, VII, or VIII, and these are applied to a support material. Aromatic compounds used here include 4,4′-MDA and isomers thereof. EP 0111238 A1 also discloses a process for catalytic hydrogenation of 4,4′-MDA, characterized in that the hydrogenation is effected in the presence of supported ruthenium in the presence of nitrates and sulfates of the alkali metals and nitrates of the alkaline earth metals. A comparable process is disclosed in EP 1366812 A1, where support materials mentioned include aluminium oxide, silicon oxide, titanium oxide and zirconium oxide.

Further processes for hydrogenation of organic compounds are disclosed by WO 2011/003899 A1 and WO 2009/090179 A1. What are disclosed here are processes for hydrogenation of aromatic amines with hydrogen in the presence of an Ru catalyst containing zirconium oxide support material inter alia.

Finally, EP 2 883 863 B1 (D1b) discloses a process and a plant for hydrogenation of 4,4′-methylenedianiline (MDA) and/or polymer MDA with hydrogen in the presence of a catalyst. A catalyst proposed here is ruthenium applied to a zirconium oxide support material. With regard to the reactor, EP 2 883 863 B1 refers to the reactor or the reactor concept of WO 2008/015135 A1 (D1a). Said WO 2008/015135 A1 discloses a continuous process and a plant for hydrogenation of diisononyl phthalate to diisononyl cyclohexane-1,2-dicarboxylate (DINCH), wherein DINP is hydrogenated as a mixture in an organic solvent with hydrogen at a pressure of up to 325 bar.

What is proposed here is series connection of two fixed bed reactors each with an immobile fixed bed. In order to dissipate the heat of reaction from the two reactors, it is proposed that a substream of the stream of matter be recirculated and thereby cooled downstream of the second fixed bed reactor. A comparable reactor concept of series-connected fixed bed reactors for hydrogenation of is also known from EP 1 566 372 B1.

This concept is disadvantageous in that the circulation of a product substream can lead to increased formation of unwanted by-products and reduces plant performance, causing increased energy costs for the recycling and cooling of the recycling stream.

As explained, the need for PACM, for example, with different proportions of the respective isomers is dependent on the intended use or the subsequent products. For example, PACM qualities with a low trans/trans content of 10% to 30% by weight are preferred in the field of amine and isocyanate crosslinkers, especially in the field of formulation of 2-component resins, and PACM qualities with a high trans/trans content exceeding 48% by weight are mainly used as regulator in polyamide compounds. The percentages by weight mentioned are based here on the PACM isomer mixture as such. The production of products with a low trans/trans content in particular is a process engineering challenge, since the thermodynamic equilibrium, as described in U.S. Pat. No. 3,636,108 A, is in the range of significantly higher trans/trans contents of up to 51.2% by weight. Furthermore, it is known from U.S. Pat. No. 2,606,925 A that the equilibrium of the PACM isomers is subsequently shifted in the direction of a higher proportion of trans/trans isomer by prolonged heat treatment.

### SUMMARY OF THE INVENTION

It is thus an object of the present invention to provide an improved plant and an improved process which is improved in terms of product conversion and energy efficiency and in particular enables the production of defined proportions of the respective isomers in the isomer mixture.

The invention includes but is not limited to the following embodiments:
1. A plant for hydrogenation of methylenedianiline (MDA; reactant1) with a hydrogen donor (reactant2), comprising a conditioning unit for the reactants, a reactor unit and a separation unit, wherein
the conditioning unit comprises (feed) conduits for reactant1, reactant2 and at least one solvent, at least one heat exchanger in at least one (feed) conduit, at least one mixer for mixing the reactants and/or at least one reactant with at least one solvent;
the reactor unit comprises at least one fixed bed reactor as main reactor with an immobile catalyst packing, wherein the at least one (first) main reactor comprises:
   a first flow pathway for the mixture of matter through the immobile catalyst packing and
   another separate closed flow pathway for a heat exchange medium outside the catalyst packing, and wherein
   the media circuit incorporates a heat exchanger;
the separation unit comprises at least: a first separation stage for substantia separation of the solvent and a second separation stage for separation of the at least one reactant and/or at least one by-product from the product,
wherein
i) a collection circuit for a closed first media circuit is included, in which at least one heat source incorporated is the heat exchanger of the main reactor and/or at least one heat exchanger of the separation unit and a first evaporator,
ii) an intermediate circuit for a closed second media circuit is included, incorporating the first evaporator, at least one compressor and a second evaporator, and where
iii) at least one distributor circuit for a closed third media circuit is included, in which at least one heat exchanger of the reactor unit, at least one heat exchanger of the conditioning unit and/or at least one heat exchanger of the separation unit is heat-exchangingly incorporated as a heat sink.
2. The plant according to Embodiment 1, wherein, in the intermediate circuit, a heat exchanger is incorporated crosswise into the incoming conduit branch and the return conduit branch, where the first conduit branch is incorporated into a first interior of the heat exchanger and the return conduit branch into the second interior of the heat exchanger.
3. The plant according to Embodiment 1, wherein the incoming conduit branch of the intermediate circuit incorporates at least two compressors.
4. The plant according to Embodiment 2, wherein at least one cross-conduit is provided in the intermediate circuit between the incoming and return conduit branch and branches off from the return conduit branch at a conduit node and enters the incoming conduit branch at a conduit node, where the branch is advantageously disposed between the heat exchanger and the first evaporator, and the inlet is incorporated into the incoming conduit branch either between the heat exchanger and the at least one compressor or between two compressors.
5. The plant according to Embodiment 1, wherein the media circuit of the collection circuit incorporates a collection tank and a pump, where a heat exchanger is disposed upstream of the collection tank in the return conduit branch and/or on the suction side of the pump in the incoming conduit branch.
6. The plant according to Embodiment 1, wherein the distributor circuit comprises a first conduit branch as a vapour-conducting feed, where the feed conduit branch incorporates at least one compressor.
7 The plant according to Embodiment 6, wherein a (bottoms) conduit leads from the second evaporator to the pressure side of the one compressor and to the suction side of a downstream further compressor, where this (bottoms) conduit incorporates a pump and/or the (bottoms) conduit or at least one branch of the (bottoms) conduit is guided from the second evaporator into the incoming conduit branch between two compressors, and the (bottoms) conduit has at least two branches, where each branch of the (bottoms) conduit is guided between two of the compressors, and where at least one branch of the (bottoms) conduit comprises a pressure regulator.
8. The plant according to Embodiment 1, wherein the distributor circuit comprises a further conduit branch for distribution or release of energy and a further conduit branch for media recycling, wherein the conduit branch for distribution incorporates at least one heat exchanger of the reactor unit, at least one heat exchanger of the conditioning unit and/or at least one heat exchanger of the separation unit as a heat sink.
9. The plant according to Embodiment 8, wherein the return conduit branch of the distribution circuit incorporates a heat exchanger so as to exchange heat.
10. The plant according to Embodiment 8, wherein at least some of the heat exchangers of the distribution circuit that are incorporated as a heat sink are connected in parallel.
11. The plant according to Embodiment 1, wherein the heat exchangers, acting as heat source, of the collection circuit are connected in series.
12. A process for catalytic hydrogenation of methylenedianiline (MDA; reactant1) with a hydrogen donor (reactant2), the process comprises: i
   mplementing a production by an industrial plant,
wherein the plant is designed according to Embodiment 1, where the main reactor is operated at a
temperature in the range from 80° C. to 150° C., and where, in the intermediate circuit, by the at least one compressor, vapour compression results in at least an increase in temperature of the medium in the incoming conduit branch of 30° C. to 120° C.
13. The process according to Embodiment 12, wherein the temperature of the reactant stream at the inlet of the main reactor is 90 to 140° C.

14. The process according to Embodiment 12, wherein the at least one main reactor is operated at a pressure in the range from 60 bar to 120 bar.
15. The process according to Embodiment 14, wherein the pressure in the main reactor is 70 to 110 bar.
16. The process according to Embodiment 12, wherein it is implemented continuously and catalytically for production of methylenebis(cyclohexylamine).
17. The process according to Embodiment 12, wherein, from time to, the start of the process after renewal or regeneration of the catalyst, to time t4, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.
18. The process according to Embodiment 12, wherein, in the distribution circuit, the pressure in the incoming conduit branch is increased in two or more stages, with an inlet pressure of 1.5 bar to 5 bar and a temperature of 100° C. to 150° C. in the first conduit branch downstream of the second evaporator and upstream of the first compressor.
19. The process according to Embodiment 18, wherein, in the distributor circuit, the pressure in the first conduit branch is increased in two or more stages, with a pressure of 3 bar to 30 bar and a temperature of 130° C. to 300° C. downstream of the last compressor and upstream of the first heat exchanger that acts as a heat sink.
20. The process according to Embodiment 12, wherein the energy introduced into the first evaporator by the collection circuit by the intermediate circuit and the at least one incorporated compressor and the heat exchanger incorporated crosswise is raised
   by at least a factor of 1.1 to 2.5 and/or
   the exit temperature of the first evaporator
   is raised by at least a factor of 1.2 to 3.0.
21 The process according to Embodiment 12, wherein the MDA (reactant1) comprises or is formed from a mixture of the following monomers: 4,4′-MDA, 2,4′-MDA and 2,2′-MDA, where the proportion of 4,4′-MDA is advantageously in the range from 75 to 98 mol %.

### BRIEF DESCRIPTION OF THE DRAWINGS

FIG. 1 shows a plant as a process flow diagram.

FIG. 2 shows a first embodiment of a two-stage expansion.

FIG. 3 shows a further embodiment of a two-stage expansion.

FIG. 4 shows a variant of the embodiment according to FIG. 3.

FIG. 5 shows a variant of the embodiment according to FIG. 3.

FIG. 6 shows a variant of the embodiment according to Figure. 3.

FIG. 7 shows a further embodiment of the reactor unit.

FIG. 8 shows a further embodiment of the reactor unit.

### DETAILED DESCRIPTION OF THE INVENTION

The object is achieved in accordance with the invention by a plant according to the features of Embodiment 1 and a process according to the features of Embodiment 12.

What is provided here is a plant for continuous catalytic hydrogenation of methylenedianiline (MDA; reactant1) with a hydrogen donor (reactant2), especially a gaseous hydrogen donor, preferably hydrogen (H2),
comprising a conditioning unit (104) for the reactants, a reactor unit, especially for synthesis of methylenebis(cyclohexylamine), especially 4,4′-diaminodicyclohexylmethane (PACM), and a separation unit, wherein
the conditioning unit comprises (feed) conduits for reactants1, reactant2 and at least one solvent, at least one heat exchanger in at least one (feed) conduit, at least one mixer for mixing the reactants and/or at least one reactant with at least one solvent,
the reactor unit comprises at least one fixed bed reactor as main reactor with an immobile catalyst packing, wherein the at least one main reactor comprises:
   a first flow pathway for the mixture of matter through the immobile catalyst packing and
   another separate closed flow pathway for a heat exchange medium outside the catalyst packing, and wherein
   the media circuit incorporates a heat exchanger;
the separation unit comprises at least a first separation stage for separation of the solvent and a second separation stage for separation of at least one reactant and/or at least one by-product from the product, wherein
i) a collection circuit for a closed first media circuit is included, in which heat source(s) incorporated are at least the heat exchanger of the main reactor and/or at least one heat exchanger of the separation unit, and a first evaporator,
ii) an intermediate circuit for a second closed media circuit is included, incorporating the first evaporator, at least one compressor and a second evaporator, and where
iii) at least one distributor circuit for a further closed third media circuit is included, in which at least one heat exchanger of the reactor unit, at least one heat exchanger of the finishing unit and/or at least one heat exchanger of the separation unit are heat-exchangingly incorporated as a heat sink.

In this context,
the collection circuit comprises a conduit branch leading to the first evaporator or collecting energy, and a return conduit branch,
the intermediate circuit comprises a conduit branch leading to the second evaporator, also called vapour conduit, and a return conduit branch from the second evaporator to the first evaporator, also called return conduit, and
the distributor circuit comprises at least one incoming conduit branch (tops or vapour conduit) from the second evaporator to the at least one heat exchanger of the separation unit and/or of the reactor unit that has been incorporated as a heat sink to the second evaporator.

In the present context, what is meant by a closed circuit is that the respective heat exchange medium can circulate only in that circuit, and in particular is not passed onward into the adjacent circuit, such that the exchange of energy with the respectively coupled adjacent circuit is effected via indirect heat transport between the circulating (circulation) media, without exchange of the respective (circulation) media. The intermediate circuit here is coupled to the collection circuit and the at least one distributor circuit. The collection circuit serves as heat source for the intermediate circuit; the intermediate circuit serves to raise the temperature level and as a heat source for the distributor circuit and the heat exchangers incorporated therein that function as a heat sink, especially the heat exchangers of the separation unit.

The liquid stream of matter coming from the separation tank is directed via a conduit to the first separation column within the first separation stage of the separation unit. Advantageously, in one embodiment of the plant, it may be the case that an expansion unit is provided in the conduit from the separation tank to the first column. This allows the reactor unit to be operated at a first, high pressure level, and the first separation stage of the separation unit at a second, lower pressure level.

The plant is preferably used for continuous catalytic production of methylenebis(cyclohexylamine) as product, especially for production of 4,4′-diaminodicyclohexylmethane (PACM), of the formula (I)

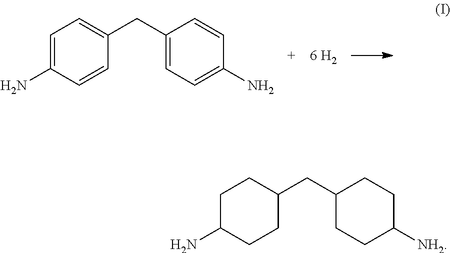 [SMILES: Nc1ccc(Cc2ccc(N)cc2)cc1 ; NC1CCC(CC2CCC(N)CC2)CC1]

With the separate postreactor, especially the adiabatic postreactor, an optimized control of the process and the plant has been enabled, whereby selectivity becomes possible because of the variance of the inlet temperature of the postreactor from the main reactor (outlet). Typically, the inlet temperature of the postreactor may be provided at the same or a slightly lower temperature, which may be up to 30° C. below the outlet temperature of the main reactor. Thus, at least temporarily, an increase in temperature in the (adiabatic) postreactor of 20° C. to 40° C., typically of 20° C. to 30° C., may be allowed, and hence the desired product quality (isomer ratio) can be specifically adjusted. In this way, a very low and a very exact proportion of trans/trans isomers in the isomer mixture can be established. The main reactor can be operated at a very low temperature level such that the trans/trans content of the PACM in the stream of matter (outlet of the main reactor) is about 13% to 20% by weight, with about 13% being achievable in the case of a new or regenerated catalyst and 20% by weight in the case of a catalyst after prolonged use (shortly before replacement/regeneration). Even though the main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation. Depending on the main reactor phase, it may be useful at least temporarily if the inlet temperature of the postreactor is 5 to 20° C. higher than that of the main reactor.

The postreactor is charged via the upstream heat exchanger with a somewhat higher temperature so as to result in the desired residual reaction and isomerization to give the final desired trans/trans ratio of 17% to 23% by weight, for example. The postreactor with the weakly exothermic residual reaction therein is operated in a largely adiabatic manner, although this should not be understood in the ideal sense. In the postreactor, the isomer content of trans/trans isomers is increased further in order to fully convert MDA. Advantageously, the concentration of MDA downstream of the postreactor is less than 1000 ppm. In a preferred process regime, a trans/trans content in the isomer mixture of 10% to 20% by weight downstream of the main reactor is attained, about 13% by weight in the case of the fresh, more active catalyst and up to about 20% by weight in an (old) laden catalyst.

As time advances, the catalyst activity decreases until replacement or regeneration is required. For this purpose, in parallel, the operating temperature is raised by controlling the cooling circuit, i.e. by cooling to a lesser degree via the heat exchanger incorporated in the cooling circuit in order to keep conversion and selectivity at a largely constant level overall. As a result, the isomer ratio is shifted, toward higher trans/trans contents in the product. It has been found here to be very advantageous that the temperature in the feed of the postreactor can be controlled autonomously by means of the upstream heat exchanger, in particular in the same sense. Thus, as the operating temperature of the main reactor rises over the service life of the catalyst, the feed temperature in the stream of matter of the postreactor is lowered.

From time t₀, the start of the process after renewal or regeneration of the catalyst, to time t₄, the end of the process determined by renewal or regeneration of the catalyst, the operating temperature of the main reactor can be increased and the temperature in the stream of matter in the inlet (feed) to the postreactor can be maintained or lowered. Advantageously, the plant is designed such that the temperature can be increased or lowered continuously and/or stepwise.

The main reactor is operated here isothermally or largely isothermally, and the postreactor adiabatically or largely adiabatically. At time to, the start of process, the catalyst in the postreactor may also have been renewed or regenerated as well as the main reactor. Advantageously, the cycle for regeneration of the postreactor is determined autonomously and independently of the main reactor, especially when the abovementioned interaction of the two reactors no longer enables the production of the desired low trans/trans content in the PACM.

Advantageously, the mixing unit is formed in two parts and comprises, for example, a mixing apparatus and a gas saturator. The mixing apparatus may especially be a dynamic or static mixer suitable for intimate association of MDA (reactant1) with the solvent. The gas saturator may especially be a small column or a tank, in which suitable internals are available for intensive dissolution of the H2 gas supplied under a high pressure in the MDA solvent stream of matter and/or for homogeneous distribution thereof prior to entry into the main reactor. The H2 gas pressure is advantageously 70 bar to 100 bar.

The term “immobile catalyst packing” or “immobile catalyst” refers to any form of a local catalyst that does not flow or move with the stream of matter, such as, in particular, catalyst beds (pellets or coated carrier bodies) or fixed installations coated with catalyst material. Advantageous internals having a catalyst coating may be, for example, grids, plates or other bodies arranged in the main reactor.

In the present context, an “XY unit” and/or “XY stage” always also means at least one corresponding apparatus, device or the like which is included in the respective unit or stage. This means, for example, a mixing unit/stage comprising this at least one mixer/mixing device.

What is meant in the present context by “heat exchange” or “heat exchanger” is always indirect heat exchange and corresponding designs with closed material and media conduits, in the absence of any explicit description to the contrary.

In the present context, essentially the following energy couplings (EC) are considered, such as integrated energy coupling or direct energy coupling, where integrated EC means integrated energy coupling, subdivided into
a. integrated material-based energy coupling (integrated material-based EC) of at least two streams of matter in a heat exchanger in indirect heat exchange, i.e. structural integration in a single heat exchanger (apparatus), meaning that, where two heat exchangers were formerly provided, both heat transfer functions of a structural unit (heat exchanger) are integrated,
b. integrated media-based energy coupling (integrated media-based EC) of at least two heat exchange media in a heat exchanger in indirect heat exchange, meaning structural integration in a single heat exchanger (apparatus).

The aforementioned ECs may be designed as direct energy coupling (direct EC) in that a serial EC or serial interconnection of at least two heat exchangers is provided.

The “condensation unit” of the first separation stage refers to a single heat exchanger or a group of heat exchangers which are used for at least partial condensation and/or cooling of the fractions of light boilers derived via the top conduit(s). A “condensation unit” referred to as such need not be a (closed) structural unit. Thus, to some degree, the term “condensation unit” and a single “heat exchanger” are also used synonymously.

The stage referred to as “first separation stage” is determined in particular, and has corresponding apparatus and conduits, in order to (specifically) separate the solvent from the product-rich stream of matter and advantageously also to return it to use in the reactor unit and/or the conditioning unit.

The first separation stage is determined in that at least 80%, ideally at least 90%, of the solvent is separated off. In an analogous manner, the stage of the separation unit referred to as the “second separation stage” means that it is thus determined, and has corresponding apparatuses and conduits, in order to separate the product from by-products and reactants, in particular MDA, and purify the product. In this case, the first and second separation stages may not be entirely strictly separated, and there may be an overlap region or an apparatus in the transition region in which both solvent and at least one by-product or at least one reactant is separated from the product. In the present context, what is meant by the “removal of solvents” in the first separation stage and “separation of at least one reactant and/or at least one by-product from the product”, where the product may especially be PACM, is that the removal/separation does not mean an absolute delimitation of the separation stages, but affects “essentially” only the substance(s) mentioned.

The liquid stream of matter coming from a flash separation tank, referred to hereinafter as separation tank (also called flash vessel in some cases), is directed via a conduit to the first separation column within the first separation stage of the separation unit. It is a feature of the separation tank that the incoming stream of matter is separated into a vapour phase (solvent, solvent-rich) and a liquid phase (solvent-depleted) by expansion (lowering the pressure) and that both phases are present in the separation tank in regular operation. The separation tank may additionally have a bottoms circulation system with integrated heat exchanger or be connected thereto in order to increase the removable vapour content beyond the pressure-related fraction by heating the liquid phase. Furthermore, a separation tank may comprise internals or random packings, in order in particular to prevent entrainment of droplets that have only been incompletely depleted of solvent, if at all. Advantageously, in one embodiment of the plant, it may be the case that a pressure control unit is provided in the conduit from the separation tank to the first column. This allows the reactor unit to be operated at a first, high pressure level, and the first separation stage of the separation unit at a second, lower pressure level. What is thus meant in the present context by separation tank (flash vessel) is a device whereby a phase separation is caused essentially by expansion. What is meant here by separation column, by contrast, is an apparatus in which a separation into a vapour phase and liquid phase takes place essentially by supply of energy, in particular by incorporating a tops circulation system in which at least a portion of the liquid condensed out is directed back into the column at the top.

What is meant in the present context by the term “reactant mixture” is the mixture of matter present on entry into the (first) main reactor, i.e. the mixture of all reactants, solvents, auxiliaries etc. in and downstream of the at least one reactor, the flowing mixture of matter in any degree of reaction or subsequent purification is referred to as “stream of matter” or “mixture of matter”, in some cases with addition of adjective descriptions such as “product-rich” or “solvent-rich”. However, the physical composition of the stream of matter at each site in the plant is also obvious to the skilled person by virtue of that site in the plant and upstream plant components, and in particular the process engineering apparatuses of the plants. The pure substances, for example the PACM product, and the LB and HB by-products, are named and identified separately. “LB” here stands for “low boilers”, a valuable mixture of matter that is separated separately from the stream of matter and from the product and has a low boiling point of about 240° C. to 290° C. Analogously, “HB” stands for “high boilers”, a valuable mixture of matter that is separated separately from the stream of matter and from the product and has a high boiling point of >350° C.

What are described in particular in the present context are a plant and a process for production of methylenebis(cyclohexylamine) as product, especially for production of 4,4′-diaminodicyclohexylmethane (PACM), by catalytic hydrogenation of methylenedianiline. The primary product here is PACM, which is hydrogenated from 4,4′-diaminodiphenylmethane (4,4′-MDA; primary fraction of reactant1), especially with the low trans/trans isomer ratio mentioned, and so the plant and the process serve, and are suitable, in particular for production of 4,4′-diaminodicyclohexylmethane (PACM) by continuous catalytic hydrogenation of 4,4′-diaminodiphenylmethane (4,4′-MDA). The additional fractions of 2,4′-MDA and 2,2′-MDA of the MDA present are converted at least in small proportions in parallel to reaction products, where these generally remain in the product mixture. In addition, other by-products can advantageously likewise be purified and separated off by this process or this plant in the second separation stage of the separation unit, especially in at least one separation column. These are regularly secondary (valuable) products, herein described as and meaning essentially high boilers (HB) and low boilers (LB).

The solvent is advantageously from the following group of substances: cyclohexane, dioxane, tetrahydrofuran (THF), cyclohexylamine, dicyclohexylamine, methanol, ethanol, isopropanol, n-butanol, 2-butanol, 2-methoxy-2-methylpropane (MTBE) or methylcyclohexane or a mixture thereof. Advantageously, the solvent, especially THF, is fed in excess into the MDA, such that the ratio of the mass flow rates of solvent, especially THF, to MDA at the inlet of the main reactor is advantageously in the range from 1.0 to 8.0, especially in the range from 2.0 to 6.0.

In an advantageous embodiment of the plant, it may be the case that the intermediate circuit incorporates a heat exchanger crosswise in the incoming conduit branch (vapour conduit) and the return conduit branch in order to raise the temperature in the incoming conduit branch.

Advantageously, the heat exchanger having a first interior, especially the vapour space, is incorporated so as to exchange heat in the incoming conduit branch between the first evaporator and the at least one, especially the first, compressor, where the return conduit branch is incorporated into the second interior of the heat exchanger, especially within the WT tubes/shell- and-tube system. In other words, the vaporous medium in the incoming conduit flows outside the heat exchanger tubes, for example, of the heat exchanger, and the largely condensed liquid medium in the return conduit branch flows within the heat exchanger tubes, for example.

In one embodiment, it may be advantageous when a cross-conduit is provided between incoming and return conduit branch and branches off from the return conduit branch at a conduit node and enters the incoming conduit branch at a conduit node, where the branch is advantageously disposed between the heat exchanger and the first evaporator, especially upstream of the expansion valve, and the inlet is advantageously incorporated either between
the heat exchanger and the at least one compressor, or
between two compressors incorporated in the incoming conduit branch to increase the pressure.

The advantage of the crosswise interconnection via the heat exchanger is that the temperature in the incoming conduit branch is raised by 2 to 10° C. by the returning medium. This interconnection serves to slightly overheat the incoming conduit branch in order to prevent condensation forming in the inlet of the compressor, which could damage it. By means of transverse introduction of liquid medium via a pressure control unit from the return conduit into the incoming conduit downstream of a compressor, especially between two compressors, it is advantageously possible to significantly increase the volume flow rate and thus the amount of energy without increasing the power/energy consumption of the forward compressor. The medium supplied via the intermediate feed is expanded at the pressure control unit and introduced as vapour. Efficiency in the incoming conduit branch is increased by about 5 to 10% by the intermediate supply.

In a further advantageous embodiment of the plant, it may be the case that the intermediate circuit, in the incoming conduit branch, incorporates a group of at least two compressors. It has been found to be advantageous to perform the desired pressure rise in multiple stages because this reduces power consumption overall since the downstream compressors have to compress an ever lower vapour volume flow rate, which means that they can be of structurally smaller design.

In a further advantageous embodiment of the plant, it may be the case that the media circuit of the collection circuit incorporates a collection tank and a pump, where a heat exchanger is disposed upstream of the collection tank and/or on the suction side of the pump. In one embodiment, the heat exchanger is integrated into the collection tank and/or the collection tank has an integrated temperature control unit. The advantage here is that overall the required temperature level and the required volume flow rate of medium can be provided in the incoming conduit branch regardless of the current energy input into the collection circuit and/or the current energy consumption in the incorporated first evaporator. The circulated heat exchange medium in the collection circuit is advantageously water or a substantially aqueous solution.

In a further advantageous embodiment, it may be the case that the distributor circuit comprises a first conduit branch as a vapour-conducting feed, where this conduit branch incorporates at least one compressor, and ideally a group of compressors comprising two to five compressors, especially comprising two or three compressors, is provided. Two or more compressors are advantageously connected in series to one another.

In this way, the temperature of the vapour coming from the second evaporator in the incoming conduit can be raised with high efficiency by 30 to 150° C., especially raised by 70 to 120° C. Overall, the distributor circuit is a water circuit, or a circuit, the heat exchange medium of which is water or an aqueous solution.

In a further advantageous embodiment of the plant, it may be the case that, with two or more evaporators in the incoming conduit branch, at least one (bottoms) conduit and/or at least one branch of a (bottoms) conduit leads from the second evaporator to the suction side of at least one compressor, with a pump incorporated in the (bottoms) conduit. Advantageously, in one embodiment, a (bottoms) conduit and/or at least a branch of a (bottoms) conduit is guided in each case between each pair of two compressors. It has surprisingly been found to be energetically advantageous overall that liquid medium from the (bottoms) conduit at a somewhat lower temperature level is fed into the incoming (vapour) conduit branch because a greatly increased mass flow rate is achieved without significantly increasing the energy requirement of the downstream compressor. Advantageously, a pressure control unit is provided in the (bottoms) conduit or the respective branch from the (bottoms) conduit in order to evaporate the liquid medium from the (bottoms) conduit via the expansion, such that the feed into the incoming conduit branch is in the form of a vaporous medium. In addition, the respective volume flow rates are controlled or, if necessary, shut down entirely by means of the pressure control units.

In other words, an advantage is that the intermediate introduction between two compressors also achieves a benefit for the second compressor stage. The vaporous medium is superheated downstream of the first compressor stage. This superheating is reduced by intermediate feeding of lower-temperature steam in spite of the material input, which reduces the volume flow rate to the second compressor stage, so as to lower the power consumption of the second compressor for further compression.

In a further advantageous embodiment of the plant, it may be the case that the (bottoms) conduit has at least two branches, with each branch of the (bottoms) conduit guided between two of the compressors, and with at least one branch of the (bottoms) conduit comprising a pressure controller, where pressure and/or temperature sensors may be provided alternatively or additionally. The pump operating in the (bottoms) conduit is advantageously disposed downstream of the second evaporator and upstream of the first branch or the conduit branches for the respective intermediate feed.

In a further advantageous embodiment of the plant, it may be the case that the recirculating conduit branch, also called (return) conduit, of the distributor circuit incorporates a heat exchanger so as to exchange heat. This heat exchanger is incorporated downstream of the conduit branch for distribution into the return conduit branch. This heat exchanger is an on-demand or control heat exchanger that can be used to ensure that medium is provided to the second evaporator at the required temperature level regardless of the energy consumption or the energy release in the conduit branch for distribution. This on-demand or control heat exchanger is not assigned to any other apparatus in the plant, i.e. is not intended to perform any other heat exchange function than to control the inflow to the second evaporator.

In a further advantageous embodiment of the plant, it may be the case that the distributor circuit comprises a further conduit branch for distribution and/or release of energy and a further conduit branch for media recycling, wherein the conduit branch for distribution incorporates at least one heat exchanger of the reactor unit, at least one heat exchanger of the conditioning unit and/or at least one heat exchanger of the separation unit as a heat sink, in particular incorporates a plurality of the respective heat exchangers. In a particularly preferred variant, each of these other conduit branches for distributing and/or releasing energy is connected in each case to one branch from the incoming conduit branch, which correlate with different pressure levels. Thus, a first branch may be disposed downstream of the first compressor, a second branch downstream of the second compressor, etc., and/or a first branch may be disposed downstream of a first pressure control unit, a second branch downstream of a second pressure control unit, etc.

This grouping of the heat exchangers by further conduit branches allows individual heat exchangers or groups of two or more heat exchangers to be fed in a controlled manner according to energy requirement and as required by the temperature level. Thus, it is advantageously possible first to discharge a heat exchanger or a group of heat exchangers with a low temperature level and/or a high exchange power in flow direction, such that the power consumption of the downstream compressors is correspondingly reduced. As required, medium in vaporous phase can be supplied from the (bottoms) conduit in the incoming conduit branch, as described above.

In a further advantageous embodiment of the plant, it may be the case that, when there is more than one return conduit branch of the distributor circuit, an on-demand and/or control heat exchanger is incorporated in at least one further return conduit branch so as to exchange heat, ideally with all return conduit branches incorporating such an on-demand and/or control heat exchanger.

In a further advantageous embodiment of the plant, it may be the case that at least some of the number of the heat exchangers in the distributor circuit incorporated as a heat sink are connected in parallel. Advantageously, the individual heat exchangers or the groups of heat exchangers connected in parallel are each controllable by open-loop and/or closed-loop control in the inlet or outlet. Controllability relates here in particular to the respective flow rates of medium, where this control is advantageously effected depending on the required temperature gradient in the respective heat exchanger or the group of heat exchangers and/or the required energy transfer. In a further advantageous embodiment, an individual heat exchanger serving as a heat sink or a group of two or more heat exchangers is not incorporated into the distributor circuit. The integration of these heat exchangers, in particular (bottom) heat exchangers of the separation columns from the separation unit, when incorporated into the distributor circuit, would have an electrical power demand for the compressors and/or another compressor which is comparable to direct electric heating of these heat exchangers. It has been found to be advantageous overall if, even in the case of an elevated energy requirement for direct electrical heating of heat exchangers of a factor of 1.2 to 1.3 compared to integration into the distributor circuit, electrical direct heating is advantageous owing to other smaller demands, such as less wear, shorter shutdown times, etc.

In a further advantageous embodiment of the plant, it may be the case that the heat exchangers, acting as a heat source, in the collection circuit are connected in series. For this purpose, the incorporated heat exchangers are advantageously incorporated into this conduit branch with a rising temperature level in flow direction of the incoming conduit branch. The central heat source used for the collection circuit is the heat exchanger incorporated in the cooling circuit of the main reactor and used to dissipate the exothermic energy from the reaction. Further heat sources used are especially the (top) heat exchangers (condensers) of the separation columns, depending on the temperature level therein in the medium of the respective heat exchanger.

The liquid stream of matter coming from the separation tank is directed via a (feed) conduit to the first separation column in the first separation stage of the separation unit. Advantageously, in one embodiment of the plant, it may be the case that an expansion unit is provided in the (feed) conduit from the separation tank to the first column. In this way, the reactor unit may be operated at a first, high pressure level, and the first separation column of the first separation stage at a second, lower pressure level, where the separation tank may be operated at an intermediate level.

Furthermore, it may be advantageous when a (forward) heat exchanger is provided upstream of the first separation column of the first separation stage, especially also downstream of the expansion unit or between the expansion unit and the first separation column. In a particularly advantageous embodiment, an energy coupling is provided in order to operate the condenser downstream of the separation tank, a condenser of a separation column of the second separation stage or the (circulating) heat exchanger in the media circuit of the at least one main reactor interconnected in heat exchange with the (forward) heat exchanger of the first separation column.

The energy coupling can be effected by conduction of media and a series interconnection of the respective heat exchangers or by integrated energy coupling in a (structurally) single heat exchanger. Integrated energy coupling has the advantage, if spatially implementable within the plant, that only a temperature gradient for heat transfer has to be overcome. It is thus possible by energy transfer to achieve a parallel rise in the (feed) stream of matter upstream of the first separation column of the first separation stage, which is at a temperature level of about 85 to 95° C. downstream of the upstream pressure control unit in the (feed) conduit. Since the main reactor, in the course of operation, is operated at a constantly rising temperature in order to compensate for the falling catalyst activity, a constantly rising amount of energy can be released to the (feed) stream of matter upstream of the first separation column in parallel. As a result, there is likewise a steady drop in the energy demand in the bottoms circuit, or the heat exchanger of the first separation column incorporated therein.

In one embodiment of the plant, it may be advantageous that the reactor unit comprises a first main reactor and at least one downstream permanent, series-connected postreactor. The particular advantage of the postreactor and its inlet-side temperature control of the stream of matter is that optimized control of the selectivity of the proportions of isomers is enabled in this way, because of the preferably higher inlet temperature that differs from the main reactor. Advantageously, the postreactor is also a fixed bed reactor, or reactor with an immobile catalyst, for example a catalyst bed or catalyst-coated internals.

Advantageously, the immobile catalyst comprises ruthenium, has been doped with ruthenium or is formed therefrom. In an advantageous process variant, especially for achieving a low proportion of trans/trans isomers in the isomer mixture, the main reactor is operated at a temperature of 90 to 140° C., ideally 95 to 135° C., as described in more detail in the context of the process according to the invention.

It has been found to be particularly advantageous when the ratio of the catalyst masses of the main reactor to the postreactor is in the range from 1.2 to 2, preferably 1.3 to 1.4 and ideally 1.35. It has been found that, surprisingly, it is sufficient to regulate the highly exothermic reaction at the start of the reaction in the main reactor by means of an intensive cooling circuit, and only to adjust the feed temperature in the inlet to the postreactor such that the moderate temperature rise of about 30 to 35° C. in the postreactor from the inlet to the outlet has only a limited and readily reproducible influence on the trans/trans isomer content in the stream of matter or in the product, as already set out.

It may be especially advantageous when the main reactor is a fixed bed reactor comprising
a first flow pathway for the reactant mixture or mixture of matter and
a further (closed) flow pathway, i.e. a media circuit for a heat exchange medium, where the second flow pathway incorporates two heat exchangers for indirect heat exchange:
   a (main) heat exchanger operating as a cooler and
   a (secondary) heat exchanger operating as a heater.

It has been found to be very effective and advantageous to operate the same further flow pathway, or media circulation, for preparation and startup of the main reactor by means of the (secondary) heat exchanger and to operate the cooling of the main reaction in producing operation by the main reactor by means of the (main) heat exchanger.

In a further embodiment of the plant, it may be advantageous that
the reactor unit comprises a further main reactor in the form of a fixed bed reactor comprising
   a first flow pathway for the mixture of matter and
   a further (closed) flow pathway, i.e. a (cooling) media circulation system for a heat exchange medium, and where a valve unit is provided upstream of the two main reactors in the (feed) conduit, by means of which the volume flow rate of the reactant mixture is divisible, conductable and/or completely switchable between the first main reactor and the further main reactor, and wherein the two main reactors are connected (in a thermally conductive manner)
   each to one heat exchanger or
   collectively to one heat exchanger
for the further (closed) flow pathway.

In this case, the two main reactors connected in series are identical or essentially identical in design. In particular, the two main reactors have such dimensions and/or corresponding internals such that the same or substantially the same mass and/or volume of catalyst is present or accommodatable.

The advantage of these two main reactors is that further thermal decoupling of the first reaction phase with very strong exothermicity, the second reaction phase with medium exothermicity and the postreaction with very low exothermicity is possible. Further advantages are that, in the case of the same plant performance, the individual main reactor has smaller dimensions and can therefore be thermally controlled more easily and more homogeneously. At the same time, plant performance is increased because, in the case of maintenance, for example a catalyst changeover, the plant does not have to be shut down completely. For this purpose, the two main reactors are interconnected in such a way that the mixture of matter can also flow through each alone, bypassing the respectively other main reactor (bypass 1).

The hydrogenation in the (isothermal) main reactor with significant cooling allows significant limitation of temperature-induced isomerization. In the (adiabatic) postreactor, a sufficient temperature level is then established in a controlled manner in the incoming stream of matter, especially via heat exchange, and hence isomerization is permitted to specifically afford an on-spec trans/trans content in the product. In a purely isothermal mode of operation of a single main reactor without a postreactor, what would at first be obtained would be excessively low trans/trans contents and a high proportion of unconverted MDA. By virtue of the option of allowing controlled adiabatic hydrogenation of the stream of matter, it is possible to advantageously control the evolution of temperature and hence isomerization in the postreactor.

In a further-improved variant, the interconnection is such that the postreactor can be bypassed in the case of operation of at least one main reactor (bypass 2), but can especially be bypassed in the case of operation of the two main reactors connected in series. In the bypass 2 interconnection variant, the main reactor through which the flow passes second in flow direction at least temporarily assumes the function of the postreactor, such that the plant can be operated without or largely without a drop in performance and/or changes in product quality, especially in the respective proportion of isomers in the isomer mixture.

In a further embodiment of the plant, it may be advantageous that at least one common heat exchanger is disposed in the conduit between the at least one main reactor and the second main reactor. This common heat exchanger is disposed in a central branch of both coolant circuits. The guiding and interconnection of the conduits here is such that, downstream of the common heat exchanger, the cooling medium is first introduced into the first of the two main reactors in which the more strongly exothermic reaction proceeds. The already heated cooling medium is then fed via a conduit into the downstream second main reactor, such that the latter is charged with a feed temperature different from the first main reactor.

The advantage is that it was observed that, surprisingly, in particular, very exact control of the first, highly exothermic reaction phase is crucial for product quality, and so it is possible to dispense with the construction work and control complexity involved in a further, completely independent second cooling circuit. Another reason for this is in particular because complete reaction can be ensured and controlled via control of the feed temperature of the postreactor connected in series downstream of the two main reactors; in particular, the desired low trans/trans isomer content can be established.

In a plant variant with improved controllability, it may be the case that a heat exchanger (post cooler) which is switchable and controllable as required is disposed in the respective (cross-) conduit of the cooling circuits by which the coolant outlet of the first main reactor is connected to the coolant inlet of the second main reactor.

The bottoms circulation system of the separation tank is operated at a temperature of 130° C. to 150° C., ideally 135 to 145° C. The great advantage of the separation tank, which is very simple in terms of construction and control, is that the boiling temperature of the mixture is likewise lowered by the lowering of pressure, such that heating in the separation tank is only necessary up to that lowered boiling temperature. Furthermore, by virtue of this measure, about 90% of the solvent present in the reactant mixture, in particular THF, is already removable. There is no need for this purpose for a tops circuit or return stream, as in a column. The stream of matter passed onward to the first separation column thus advantageously has only a remaining solvent concentration of about 20% to 40% by weight, ideally 25% to 35% by weight. It is thus possible for the first separation column to be of smaller design, and operable in a more energy-saving manner owing to the lower mass of the stream of matter.

In this way, the temperature of the stream of matter is additionally lowerable more quickly, and so any rise in the proportion of trans/trans isomers of the PACM in the stream of matter is prevented or reduced.

The solvent-conducting (return) conduit is connected to the conditioning unit and/or to at least one suitable collection tank.

The invention further encompasses a process for continuous catalytic hydrogenation of methylenedianiline (MDA; reactant1), especially 4,4′-diaminodiphenylmethane, with a hydrogen donor (reactant2), in particular a gaseous hydrogen donor, preferably hydrogen (H2), wherein the production is effected by means of an industrial plant, wherein the plant is designed according to at least one of the working examples and variants described herein, and wherein the main reactor is operated at a temperature in the range from 80° C. to 150° C., and wherein, in the intermediate circuit, by means of the at least one compressor, vapour compression results in at least an increase in temperature of the medium in the incoming conduit branch of 30° C. to 120° C., ideally of 50° C. to 90° C.

In a further embodiment of the process, a further advantage may be that the at least one main reactor is operated at a pressure in the range from 60 bar to 120 bar, ideally in the range from 70 to 110 bar. In a further advantageous process regime, it may be the case that the pressure in the main reactor is 70 to 100 bar, ideally 80 to 90 bar. Particularly preference is given to a pressure of about 85 to 90 bar.

In a preferred embodiment of the process, it may be the case that continuous catalytic production of methylenebis(cyclohexylamine) is effected, especially production of 4,4′-diaminodicyclohexylmethane (PACM), preferably 4,4′-diaminodicyclohexylmethane (PACM) with low proportions of trans/trans isomers. In a preferred process variant, continuous catalytic production of methylenebis(cyclohexylamine) is effected, especially production of PACM, of the formula (I)

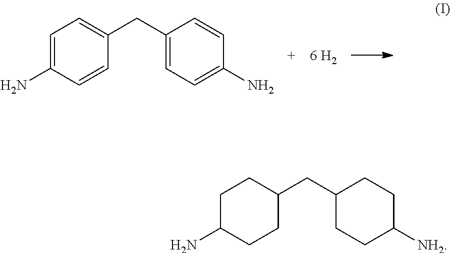 [SMILES: Nc1ccc(Cc2ccc(N)cc2)cc1 ; NC1CCC(CC2CCC(N)CC2)CC1]

In a further embodiment of the process, a further advantage may be that, from time to, the start of the process after renewal or regeneration of the catalyst, to time t4, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.

With the plant variant mentioned, it is thus possible for the process to continue until attainment of a trans/trans content in the PACM of 17% to 25% by weight over time.

Even though the main reactor is referred to in the present context as being “isothermal”, this ideal state is only achieved to a limited degree in industrial use, and so a temperature gradient of about 5 to 10° C. in radial direction and also in flow direction develops within the main reactor owing to incomplete heat dissipation.

In a further embodiment of the process, a further advantage may be that, in the distributor circuit, a multistage increase in pressure is effected in the incoming conduit branch, giving an inlet pressure of 1.5 bar to 5 bar and a temperature of 100° C. to 150° C. downstream of the second evaporator and upstream of the first compressor.

In a further embodiment of the process, a further advantage may be that, in the distributor circuit, a multistage increase in pressure is effected in the first conduit branch, giving a pressure of 3 bar to 30 bar and a temperature of 130° C. to 300° C. downstream of the last compressor and upstream of the first heat exchanger acting as a heat sink.

In a further embodiment of the process, a further advantage may be that the energy introduced into the first evaporator by the collection circuit by means of the intermediate circuit and the at least one incorporated compressor and any heat exchanger incorporated crosswise is raised
by at least a factor of 1.1 to 2.5 and/or
the exit temperature of the first evaporator
is raised by at least a factor of 1.2 to 3.0.

In a further embodiment of the process, a further advantage may be that the MDA (reactant1) comprises a mixture of the following monomers: 4,4′ MDA, 2,4′ MDA and 2,2′ MDA, where the proportion of 4,4′ MDA is advantageously in the range from 75 to 98 mol %, ideally 85 to 95 mol %, preferably 90 mol %. The proportion of 2,4′ MDA in the reactant mixture is advantageously 7 to 15 mol %, preferably 8 to 12 mol %, ideally 9 to 10 mol %. The proportion of 2,4′ MDA in the reactant mixture is advantageously 7 to 15 mol %, preferably 8 to 12 mol %, ideally 9 to 10 mol %.

Ideally, the isomer content of trans/trans-PACM in the product is in the range from 15% to 30% by weight, ideally 16% to 25% by weight.

Overall, it is advantageous when the reaction step in the main reactor is implemented isothermally or largely isothermally and the reaction step in the postreactor is implemented adiabatically or largely adiabatically. At time to, the start of process, the catalyst in the postreactor may also be in renewed or regenerated form as well as the main reactor. Advantageously, the cycle for regeneration or renewal of the postreactor is determined autonomously and independently of the main reactor, especially when the abovementioned interaction of the reaction components in the two reactors no longer enables the production of the desired low trans/trans content in the PACM.

In an advantageous embodiment of the process, it may be the case that the temperature of the reactant stream at the inlet of the main reactor is 90 to 140° C., ideally 100 to 135° C.

In a further advantageous embodiment of the process, it may be the case that
the temperature at the main reactor inlet corresponds essentially to the temperature at the postreactor inlet, where essentially means a range or difference of +/−10° C. and/or the pressure at the main reactor inlet corresponds essentially to the pressure at the postreactor inlet, where essentially means a range or difference of +/−5 bar.

Overall, all aspects, advantages and executions relating to plants or mentioned in association with the description thereof are also intended to be applicable identically or analogously to the process, and vice versa, unless stated otherwise and/or there is a technical impossibility associated with analogous application.

The solution according to the invention is described in detail hereinafter with reference to working examples.

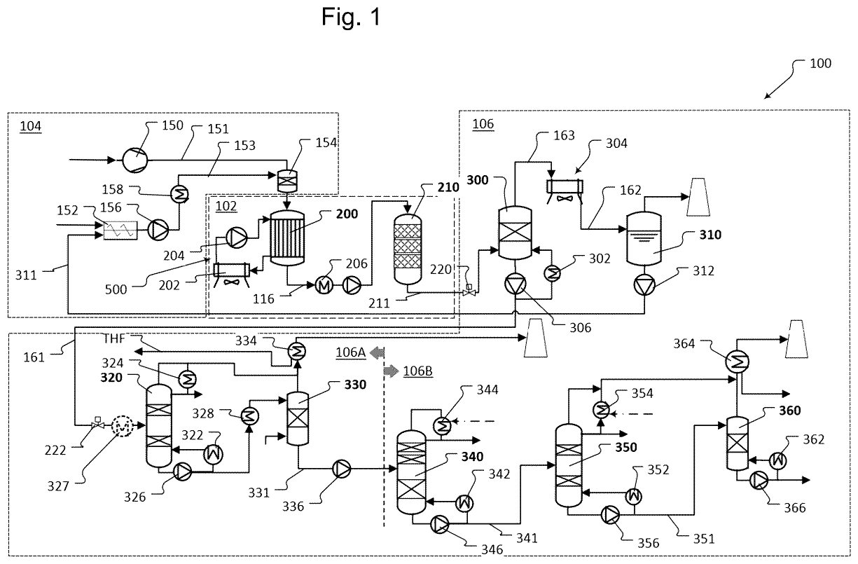

FIG. 1 shows the plant 100 for continuous production of 4,4′-diaminodicyclohexylmethane (PACM) by hydrogenation of methylenedianiline (MDA; reactant1), especially 4,4′-diaminodiphenylmethane, with a hydrogen donor (reactant2), where the hydrogen donor is supplied in the form of gaseous hydrogen (H2). The plant 100 comprises a conditioning unit 104 for the reactants, a reactor unit 102 and a separation unit 106.

The conditioning unit 104 is framed by dashed lines and comprises (feed) conduits for the reactant1 and the hydrogen (reactant2), and also the solvent. In addition, the conditioning unit comprises a compressor unit 150, referred to hereinafter as compressor in the conduit 151 supplying the hydrogen, a mixer 152 in the conduit supplying the solvent and reactant1. Additionally disposed in the conduit 153 supplying the mixture of reactant1 and solvent are a pump 156 and a heat exchanger 158. The conduits 151, 152 open into a mixing vessel 154 disposed upstream of the main reactor 200. The mixing vessel 154 serves for intensive mixing of the reactants, and its outlet forms the inlet for the main reactor 200.

The reactor unit 102 is framed by dashed lines and comprises essentially the main reactor 200, a cooling circuit 500, a postreactor 210 and a heat exchanger 206 in the feed conduit to the postreactor 210. The cooling circuit 500 incorporates a pump 204 and a heat exchanger 202, where the circulating coolant in the main reactor 200 flows around the catalyst material-filled carrier elements. In the example shown, the flow direction toward the catalyst material-filled carrier elements is in cocurrent direction. The postreactor 210 is connected via the conduit 211 to the separation unit 106, i.e. the first separation stage thereof.

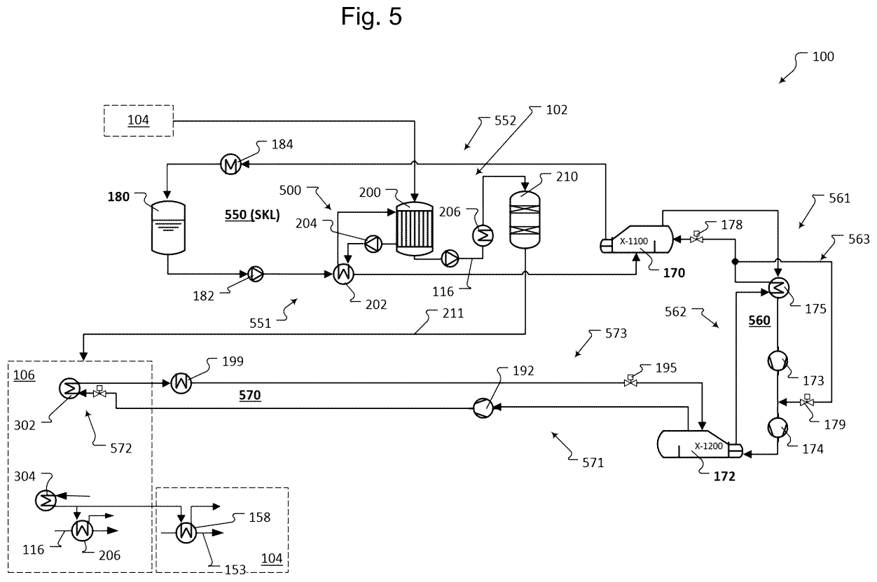

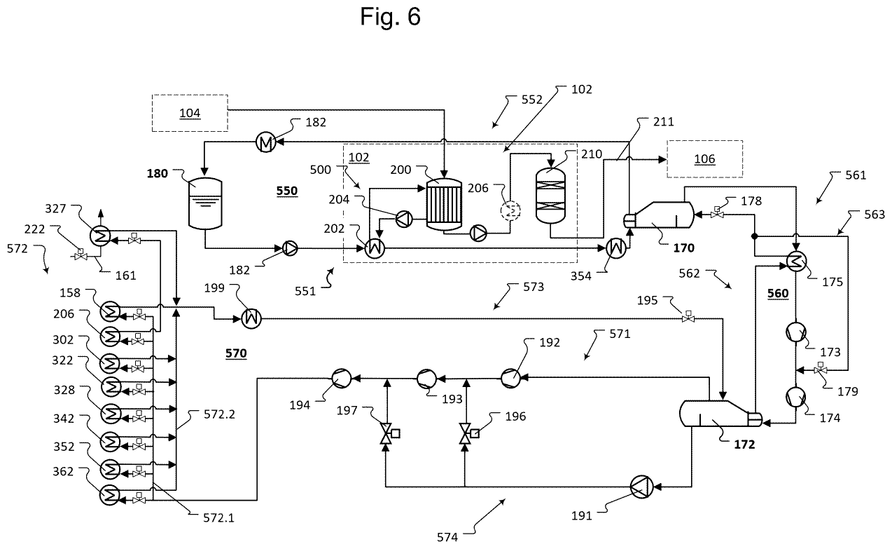

The heat exchanger 202 in the cooling circuit 500 is shown as an air-cooled heat exchanger 202, but may also be alternatively designed in order to control the temperature of the cooling medium in the cooling circuit 500 in indirect heat exchange, for example by means of a flowing cooling medium, such as an oil, water or a brine. The cooling circuit 500, in a plant variant which is not shown, also incorporates a heat exchanger, analogously to the heat exchangers 208, 209 in FIGS. 5 and 6. The latter is operated with a heating medium and serves, in the step of starting up the main reactor 200, to adjust the temperature of the main reactor 200. In the plant and process example shown, in which one aim is to achieve a minimum trans/trans isomer ratio of about 17% to 23% by weight, the main reactor 200 filled with fresh or regenerated catalyst is preheated to a temperature of about 90° C. by means of the heat exchanger 208. The main reactor 200 is operated at a pressure of 87 to 88 bar.

The separation unit 106 is framed by dashed lines and comprises a plurality of separation apparatuses for separating the solvent, especially in a first separation stage, and the PACM product, especially in a second separation stage, from the rest of the reactant and by-products. The first separation stage (not displayed) comprises a separation tank 300 (flash vessel) to which a bottoms circulation system is connected, incorporating a heat exchanger 302 and a pump 306. The top outlet of the separation tank 300 is connected to a heat exchanger 304, a condenser, downstream of which is provided a collecting vessel 310 for the solvent. By means of the heat exchanger 304 (condenser), about 80% of the solvent, THF in this case, is condensed out and could be collected or returned. In the flash stage, an energy requirement of about 1400 kW is required for the heat exchanger 302 in the bottoms circulation system of the separation tank 300.

The condenser 304 is shown as a heat exchanger in the design of an air-cooled apparatus, but may also alternatively be designed to at least partly condense and to cool the vaporous stream of matter at the outset in indirect heat exchange, for example by means of a flowing cooling medium such as cooling water or a medium suitable for thermal integration.

The amounts of energy specified herein are calculated for a plant output of the PACM product in the synthesis reaction mentioned of about 3.37 t/h, and a by-product output of about 0.4 t/h of HB and about 0.05 t/h of LB. The ratio of the mass flow rates of THF to MDA was 4.2 to 4.5.

In addition, the first separation stage of the separation unit 106 for further removal of the solvent comprises a first separation column 320 and a second separation column 330, where the second separation column 330 takes the form of a stripping column. For this purpose, nitrogen (N2) is advantageously used as a stripping medium, which is passed through the column in countercurrent to the stream of matter. By means of the pump 306 disposed in the bottoms outlet of the separation tank 300, the solvent-depleted stream of matter can be diverted via the conduit 161 to the first separation column 320. An expansion unit 222 is provided in the conduit 161 and is designed as a controllable valve in the example shown. The stream of matter is introduced into the first separation column 320 via a central inlet as shown. Additionally disposed upstream of the first separation column 320 is a (forward) heat exchanger 327 (shown by dashed lines), which constitutes an option for heating of the first separation column 320.

In the first separation column 320, the solvent concentration is reduced from 30% by weight in the feed via the product-rich stream of matter from the conduit 161 to about 2% by weight of residual solvent.

This first separation column 320, equipped with (structured) packings, is connected to a bottoms circulation system incorporating a heat exchanger 322 and a pump 326. In addition, a tops circulation system is disposed at the column top of the first separation column 320, incorporating a heat exchanger 324 designed as a condenser. Two outlets for the solvent-rich stream of matter lead out of the tops circulation system, with one of the two outlets leading into the tops outlet of the downstream separation column 330 (stripping column).

In particular, the solvent separated off in the first separation stage is conductable via the conduit 311 into a collection tank (not shown) and/or the mixer 152 of the conditioning unit 104. In the conduit 211 leading from the postreactor 210 to the separation tank 300, there is an expansion unit 220, designed in the present example as a controllable valve.

The product-rich stream of matter having a residual content of about 2% by weight of solvent (THF) is directed from the bottoms outlet of the first separation column 320 via the conduit 321 into the top of the second column 330, the stripping column. This conduit 321 incorporates a heat exchanger 328. The second column 330 has at least one internal packing, where a gas feed for an inert gas (stripping gas) in particular is disposed below the packing, such that the introduced product-rich stream of matter flows through the second column 330 in countercurrent principle to the stripping gas and is further depleted of solvent. In the plant example shown, nitrogen (N2) is used as stripping gas. The solvent-rich vapour from the second separation column 330 is directed via the tops outlet into a condenser 334, together with the solvent-rich vapour from the tops outlet of the first separation column 320. The solvent stream condensed out in the heat exchanger 334 is returned to the conditioning unit 104, wherein the uncondensable portion is led off from the heat exchanger 334 and, for example, fully thermally oxidized.

The product-rich stream of matter is fed from the bottoms outlet of the second separation column 330 to the third separation column 340 via the conduit 331, incorporating the pump 336. The second separation stage 106B of the separation unit 106 serves in particular to separate the LB and/or HB by-products from the PACM product.

The second separation stage comprises essentially three separation columns, where the third separation column 340, the first of the second separation stage, is supplied centrally with the stream of matter. The third separation column is connected to a bottoms circulation system incorporating a heat exchanger 342 and a pump 346. The product-rich stream of matter is directed via the conduit 341 from the bottoms outflow to the fourth separation column 350. In addition, the third separation column 340 is connected to a tops circulation system incorporating a heat exchanger 344. Condensed LB is led off as the first by-product from this tops circulation system.

The product-rich stream of matter is fed centrally to the fourth separation column 350 via the conduit 341. The fourth separation column 350 is connected to a bottoms circulation system incorporating a heat exchanger 352 and a pump 356. In addition, the fourth separation column 350 is connected to a tops circulation system incorporating a heat exchanger 354 in the form of a condenser. The product-rich stream of matter is discharged from the tops circulation system as condensate via the tops outlet. In the present case, according to the example shown, the uncondensed vapour stream and/or gas stream is introduced into the tops discharge of the downstream fifth separation column 360. Subsequently, product is condensed further and discharged via a further heat exchanger 364 (condenser). The stream of matter with a low level of product is discharged via the bottoms outlet of the fourth separation column 350 via the conduit 351 and introduced into the top portion of the fifth separation column 360. This stream of matter is highly enriched with HB, a second by-product. The fifth separation column 360 is connected to a bottoms circulation system incorporating a heat exchanger 362 and a pump 366. Furthermore, the separation column 360 has a tops outlet which leads to the aforementioned heat exchanger 364 in the form of a condenser. In this condenser 364, a further product-rich stream of matter is condensed out as condensate and discharged, with gaseous discharge of the uncondensable portion. In particular, the latter may subsequently be fully oxidized.

With regard to the stream of matter, the reactor unit 102 is operated at a first, high pressure level of about 60 to 120 bar, the first separation stage 106A of the separation unit 106 is operated at a second, low pressure level of 4 to 12 bar, and the first separation column 320 and the second separation column 330 are operated at a third, slightly elevated pressure level of 1.05 to 2.5 bar. In the example shown, the first pressure level is 80 to 90 bar, the second pressure level is 4.5 to 7.5 bar and the third pressure level is 1.1 to 1.2 bar.

With direct temperature control of the main reactor 200 via the cooling circuit 500 in series with the uncooled postreactor 210, surprisingly, a reduced energy requirement compared to the related art and a simplified, more stable process regime has been demonstrated. Without being tied to any specific interpretation, this success is apparent in that the cooling circuit 500 only has to be specifically designed for the very vigorous initial reaction for dissipation of the exothermic heat of reaction, while the still significant postreaction has to be adjusted via the feed temperature by means of the upstream heat exchanger 206 alone. Because the reaction in the postreactor 210 is already highly attenuated, the stream of matter is only heated by about 5 to 15° C. and can be discharged into the separation tank 300 at this level without any problems.

All in all, the figures show internals such as packings, separation planes, support elements etc. in the apparatuses such as reactors, separation columns, vessels etc. by means of the corresponding symbols. These each indicate advantageous embodiments, with regard to the number, type and/or relative position to the respective feed conduit or discharge conduit. For example, the illustration of first separation column 320 thus indicates that, advantageously, the (feed) stream of matter is introduced via the conduit 161 in such a way that at least one (theoretical) separation plane is present in each case between the tops circulation system and the bottoms circulation system. The determination of the specific type and/or number of separation planes is known to the skilled person and can be varied or provided for in an appropriate manner.

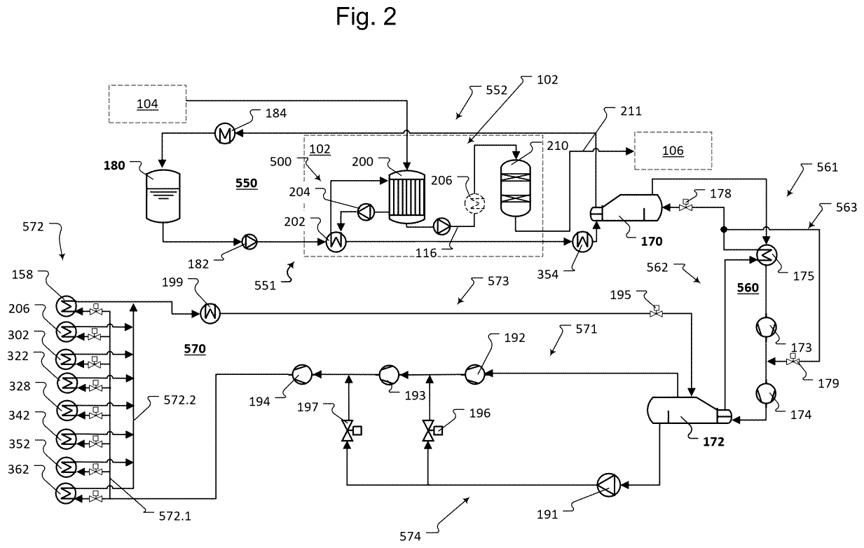

FIG. 2 shows a first embodiment of an energy circuit, wherein three circuits are provided, namely a collection circuit 550, an intermediate circuit 560 and a distributor circuit 570. The collection circuit 550 comprises a collection tank 180, a first conduit 551, an evaporator 170 and a second conduit 552, where the first conduit 551 connects the collecting tank 180 to an inlet of a heat exchanger portion (WT portion) of the evaporator 170 and the second conduit 552 connects an outlet of the WT portion of the evaporator 170 to the collecting tank 180. It is thus only the WT portion of the evaporator 170 that is part of the collection circuit 550.

The first conduit 551 incorporates the heat exchanger 202 of the coolant circulation 500 of the main reactor 200 and the heat exchanger 354 from the tops circulation system of the fourth separation column 350 as heat sources, and a pump 182. In the second conduit 552, in the example shown, the feed to the collection tank 180 incorporates a heat exchanger 182 which is used essentially to set the required feed temperature (cooling) of the collection tank 180 and/or the temperature in the first conduit 551 as required and for control reasons, in order to ensure the temperature gradient for the required cooling of the incorporated heat exchangers 202, 354. The first conduit 551 can also be regarded as a collection or feed conduit and the second conduit 552 as a return conduit. The flowing medium provided in the circuit in the example shown is water, although it would also be possible to use a brine or an oil.

In an advantageous manner, the collection circuit “collects” multiple heat sources in order to transfer the amount of energy thus collected from the medium circulated, here water in particular, to the first evaporator. This significantly reduces design complexity compared to a direct interconnection of the heat exchangers in question.

The intermediate circuit 560 comprises the aforementioned evaporator 170, a first conduit 561, two compressors 173, 174, a (forward) heat exchanger 175, a further evaporator 172, a further conduit 562 and an expansion unit 178. The first evaporator 170 incorporates the boiler portion (K portion), part of the intermediate circuit 560, and the second evaporator 172 incorporates the WT portion, meaning that the heat exchange medium in the intermediate circuit 560 flows through it. The (forward) heat exchanger 175 is interconnected such that it is integrated in the second conduit 562 downstream of the second evaporator 172 and in the feed to at least one of the compressors 173, 174 in the first conduit 561. In addition, a (bridge) conduit 563 is provided, via which the heat exchange medium can be introduced into the first conduit 561, branching off from the second conduit 562. The conduit node for the branch of the (bridge) conduit 563 is downstream of the heat exchanger 175 in the second conduit 562, and the inlet into the first incoming conduit is between the two compressors 173, 174. The heat exchange medium used is advantageously a more volatile medium compared to water, such as, in particular, an alcohol having 1 to 6 carbon atoms, especially methanol, 2-propanol, butanol or n-butanol. The expansion unit 178 is disposed upstream of the inlet of the second conduit 562 into the K portion of the first evaporator 170.

The evaporated medium, in this case methanol, is conducted at a pressure of about 2.6 bar in the first incoming conduit and superheated in two compression stages. The pressure is raised to about 9.5 bar by the compressors 172, 174, as a result of which the media stream is superheated and has a temperature of about 177° C. downstream of the second compressor 174. The media stream is cooled in the WT portion of the second evaporator 172 such that the medium condenses out and is present as a liquid phase.

In addition to the possibility of significantly raising the energy level, another advantage of the intermediate circuit is that the heat exchange medium itself constitutes an additional degree of freedom. This allows the working medium to be selected in such a way that it is optimally suited to the process-related temperatures of the collecting circuit and the supply circuit and leads to minimal compression costs for the operation of the compressors.

The second evaporator 172, the WT portion of which is incorporated into the intermediate circuit 560, is connected to the distributor circuit 570 incorporating the K portion of this evaporator 172. The distributor circuit 570 comprises a first, incoming conduit branch 571, a second distributor conduit branch or conduit section 572 and a third return conduit branch 573, also called (return) conduit. A tops discharge of the evaporator 172 leads into the first conduit branch 571, where, in the exemplary embodiment shown, three series-connected compressors 192, 193, 194 are incorporated into the first conduit branch 571. Furthermore, the distributor circuit 570 comprises a (bottoms) conduit 574 which opens from a bottoms outlet of the evaporator 172 via two conduit nodes into the first conduit branch 571, where a pump 191 is incorporated in the (bottoms) conduit 574. The first introduction of heating medium is effected via the first conduit node between the first compressor 192 and the second compressor 193; the second introduction of heating medium is effected between the second compressor 193 and the third compressor 194. A controllable valve 196, 197 is disposed in each case upstream of the respective, unidentified conduit nodes.

The distributor conduit section 572 incorporates heat exchangers to be supplied as heat sinks, the heat exchangers 158, 206, 302, 322, 328, 342, 352 and 362 that are connected in parallel to one another. A central distributor conduit 572.1 leads to the heat exchangers, and a central collection conduit 572.2 is fed by all heat exchangers and leads into the (return) conduit 573. The distributor conduit section 572 is connected to the K portion of the second evaporator 172 via the (return) conduit 573. This (return) conduit 573 incorporates a heat exchanger 199 and a pressure control unit 195, by means of which the final pressure of the third compressor 194 of 20 bar is released again to the level of the K portion of the second evaporator of about 2.5 to 3 bar. The (return) conduit 573 is connected to an inlet of the K portion of the evaporator 172.

The two evaporators 170, 172 are so-called kettle-type evaporators, which have a heat exchanger portion (WT portion) which is closed to a first flowing medium and a tank portion (K portion) which is open to another flowing medium. The WT portions each have an inlet and an outlet, where the heat exchange medium is guided in closed channels or pipes, for example at least one shell- and-tube system. The K portions each have at least one inlet and one (tops or vapour) outlet each, where the second evaporator 172 also has a (bottoms) outlet. In particular, medium introduced via the at least one (bottoms) inlet is heated by means of the respective WT portion or the associated heat exchanger and at least partly evaporated. The respective K portion may be in two parts or have two subspaces. There is a central K portion here, into which the heat exchanger of the WT portion also protrudes and the energy input into the K portion takes place. The further subspace is disposed in a lateral or outer K portion. This may advantageously, but not necessarily, be formed by internals as a zone calmed with respect to the liquid medium and/or is determined in that the heat exchanger of the WT portion does not protrude into this subspace.

The water-conducting distributor circuit is at a temperature of 130° C. and 2.7 bar at the incoming conduit branch immediately downstream of the second evaporator 172. Downstream of the first compressor 192 the pressure is 5.4 bar, which is achieved with a power consumption of 238 KW, downstream of the second compressor 192 the pressure is 10.8 bar, which is achieved with an electrical power consumption of 295 KW, and downstream of the third compressor 193 the pressure is 20 bar at a temperature of 250° C., achieved by further electrical power consumption of 250 KW for the third compressor 193.

For supply of the heat exchangers shown in FIG. 2, which are in particular the bottom heat exchangers of the separation columns, it is necessary to provide a very high temperature level, and so the circulating medium has to be cooled again via the demand and control heat exchanger 199.

In the example shown, the required cooling capacity is about 368 KW, which has to be provided in the (return) conduit 573 upstream of the pressure control unit 195 or prior to entry into the second evaporator 172.

A significant advantage of the distributor circuit can be considered to be that a central steam circuit has been created and hence steam can be generated centrally and distributed to all heat exchangers that operate as consumer (heat sink), instead of directly interconnecting heat sources and heat sinks. Furthermore, the distributor circuit as a central steam circuit, if required (for example when starting up the plant), can be fed at least temporarily by means of an alternative heat or steam source.

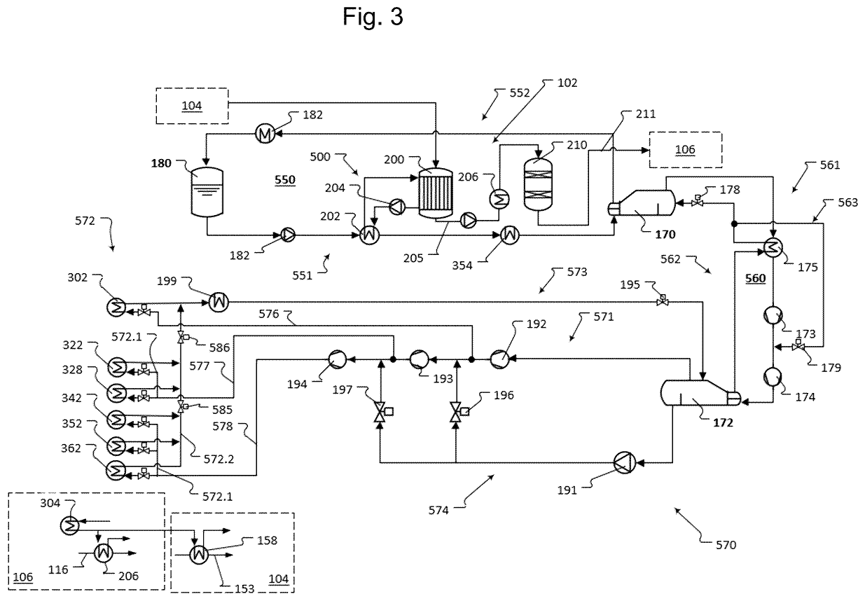

FIG. 3 shows an embodiment in which the collecting circuit 550 and the intermediate circuit 560 are formed analogously to FIG. 2. The distributor circuit 570 differs from the embodiment according to FIG. 2 in that the conduit section 572 via which energy is distributed to the heat exchangers operating as heat sinks is divided into three subsections, the three subsections being connected in parallel to one another:
a high-pressure subsection (HP subsection), connected
downstream to the third compressor 194,
mid-pressure subsection (MP subsection) connected
downstream to the second compressor 193, and
a low-pressure subsection (LP subsection), connected
downstream to the first compressor 192.

What is meant here by “LP subsection/circuit” is that this subsection or circuit incorporates “fewer compressors and/or lower compressor power” than the HP subsection/circuit, and that the possible final pressure and hence also the final temperature (without further heat exchange) is lower than in the HP subsection/circuit due to the number and/or the design of the compressors in the LP subsection/circuit. In other words, a lower pressure of the vapour generated than in the HP subsection/circuit is sufficient to fulfil the heating functions in the LP subsection/circuit at a lower temperature level. Similarly, the pressure in the MP subsection/circuit is between the LP and HP subsection/circuit.

The HP subsection/circuit in the present context means the subsection/circuit of the distributor circuit in which the maximum possible final pressure and hence generally also the highest final temperature in the distributor circuit is generatable in the medium (within the distributor circuit), which is possible owing to the power and/or number of (especially series-connected) compressors and/or the compressor output possible therewith.

Each subsection has a dedicated distributor conduit 572.1 and is connected to the evaporator 172 via an incoming conduit or conduit branch 576, 577 and 578 as branch from the incoming conduit branch 571. Furthermore, a common collecting conduit 572.2, into which each subsection feeds, leads into the return conduit branch 573. The heat exchangers within each of these subsections are connected in parallel to one another, if two or more heat exchangers are included.

In this case, the conduit node with which the first (output) conduit 576 branches off from the central conduit branch 571 is disposed between the first compressor 192 and the second compressor 193, the conduit node with which the second (output) conduit 577 branches off from the central conduit branch 571 is disposed between the second compressor 193 and the third compressor 194, and the third (output) conduit 578 which constitutes the last portion of the central conduit branch 571 is connected downstream of the third compressor 194. In this case, it is solely the first (output) conduit 576 that supplies the heat exchanger 302, the LP subsection of the conduit section 572, the second (output) conduit 577 that supplies the two parallel-connected heat exchangers 322, 328, the MP subsection of the conduit section 572, and the third (output) conduit 578 that supplies the three parallel-connected heat exchangers 342, 352, 365, the HP subsection of the conduit section 572. Thus, the three (output) conduits 576, 577 and 578 each have different pressure levels and different temperature levels. Liquid medium is fed into the central conduit branch 571 via the (bottoms) conduit 574 or via the branches, analogously to the embodiment of FIG. 2, where the conduit nodes of the three (output) conduits are disposed in flow direction upstream of the conduit node of the respective inlet from the (bottoms) conduit 574.

The lower subsection in the illustration, incorporating the heat exchangers 342, 352, 362, is kept via the pressure control unit 585 in the collecting conduit 572.2, and the central subsection incorporating the heat exchangers 322, 328 is kept at an autonomous pressure level via the pressure control unit 586 in the collecting conduit 572.2. Stepwise expansion thus takes place in the collecting conduit 572.2.

The three subsections of the conduit section 572, by means of which the energy is distributed to heat exchangers operating as heat sinks, lead into a common (return) conduit 573 and into the evaporator 172.

The advantage of this embodiment variant over that from FIG. 2 is the significantly lower energy requirement for the second and third compressors 193, 194.

The water-conducting distributor circuit is likewise at a temperature of 130° C. and 2.7 bar at the incoming conduit branch 571 immediately downstream of the second evaporator 172. Downstream of the first compressor 192 the pressure is 5.4 bar, which is achieved with a power consumption of likewise 238 KW, downstream of the second compressor 194 the pressure is likewise 10.8 bar, although this is achieved with an electrical power consumption of only 145 KW, and downstream of the third compressor 194 the pressure is analogously 20 bar at a temperature of 250° C., but achieved by electrical power consumption of only 115 KW for the third compressor 194. This advantage arises because the in the second and third compressors 192, 193 is greatly reduced. In addition to the energy benefit, this means that the second and third compressors 193, 194 may be of significantly smaller design, which also generally likewise improves maintenance and operation and/or installation.

For supply of the heat exchangers shown in FIG. 3, which are in particular the bottom heat exchangers of the separation columns, it is necessary to provide a very high temperature level, and so the circulating medium has to be cooled again via the demand and control heat exchanger 199. In the example shown, the required cooling capacity is about 368 KW, which has to be provided in the (return) conduit 573 upstream of the pressure control unit 195 or prior to entry into the second evaporator 172.

In principle, the LP subsection incorporating the heat exchanger 302 could also incorporate the heat exchangers 206 of the reaction unit 102 and/or the heat exchanger 158 of the conditioning unit 104.

In the example shown in FIG. 3, the heat exchangers 304 (condensers) for energy coupling as heat source are coupled to the two heat exchangers 206, 158 operating as heat sinks. In this case, the heat exchanger 206 is incorporated in the feed to the postreactor 210 (not shown), and heat exchanger 158 in the (feed) conduit 153 upstream of the main reactor 200. This energy coupling option of a direct thermal coupling is shown bottom left in FIG. 3. For better understanding, the heat exchange 206 and the conduit 116 are thus shown twice in this FIG. 3. This selective direct interconnection and distribution of the heat from the condenser 304, compared to the incorporation of the heat sinks in the distributor circuit 570, for example according to FIG. 2 or FIG. 3, as described above, results in an increase in efficiency of 30% to 40%, especially when at least one energy coupling is an integrated material-based EC, in that heat exchanger 304 is coupled to the (forward) heat exchanger 158 and/or the (feed) heat exchanger 327 (not shown; similarly to FIG. 6) upstream of the first separation column 320 of the first separation stage 106B.

In this case, “increase in efficiency”, unless stated otherwise, means a lower energy consumption. The reference point will be apparent from the context and may be based on the plant, the plant section respectively described or the improved apparatus, for example via integration of two heat exchangers into a single one.

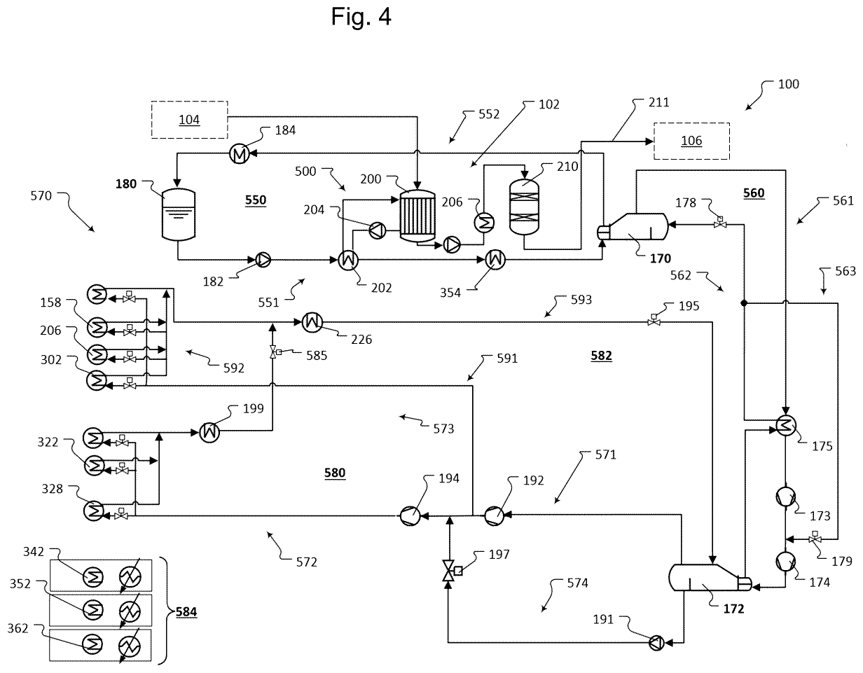

The working example as shown in FIG. 4 can be regarded as an alternative to or improvement over the working example of FIG. 3 because the HP subunit has been resolved in an alternative manner. In the working example of FIG. 4, the distributor circuit 570 comprises two part-circuits. These part-circuits are a mid-pressure distributor circuit 580 (MP distributor circuit) and a low-pressure distributor circuit 582 (LP distributor circuit). The MP distributor circuit 580 is substantially similar to the embodiment of the MP subsection in FIG. 3. This is disposed downstream of the second compressor 194, and there is likewise also provision only of an intermediate feed of a media stream via the (bottoms) conduit 574 between the two compressors 192, 194. In the branching conduit of the LP distributor circuit 582, the pressure downstream of the second compressor 194 is about 8 to 12 bar, in the present case about 10.8 bar. The LP distributor circuit 582 is substantially analogous to the embodiment of the LP subsection of FIG. 3. This is disposed downstream of the first compressor 192. In the branching conduit 591 of the LP distributor circuit 582, the pressure is about 5 to 6 bar, in the present case 5.4 bar.

The WT portion of the evaporator 172 is incorporated analogously into the intermediate circuit 560, in which there is circulation of a volatile medium, for example methanol or butanol.

The LP distributor circuit 582 comprises a first, incoming conduit branch 591, a second distributor conduit branch 592 (also called conduit section 592), which opens into a central return conduit branch 593 (also called (return) conduit 593). As explained, the incoming conduit branch 591 incorporating the heat exchangers 158, 206, 302 and any further heat exchangers as energy sink in a parallel connection branches off from the conduit branch 571 downstream of the first compressor (192). The (return) conduit 593 forms the common return conduit for the MP and the LP distributor circuit 580, 582 toward the K portion of the second evaporator 172. The (return) conduit 593 incorporates a heat exchanger 226, by means of which a requisite return temperature for the operation of the evaporator 172 and/or warm-up steps before or on startup of operation of the plant 100 or of the process can be implemented. Furthermore, a pressure control unit 195, especially one that is controllable, is incorporated into the (return) conduit 593 in order to ensure the lower pressure level in the K portion of the evaporator 172 of about 2.5 to 3.5 bar at about 125 to 130° C.

In order to keep the efficiency of the plant and of the three or four interacting circuits at an advantageous level overall, one heat exchanger group 584 (WT group 584) is not incorporated and is advantageously electrified. The heat exchangers 342, 352, 362 of the three associated bottoms circuits of separation columns are thus heated by an electric heater without a conduit connection for a flowing heat exchange medium. The motivation for this is that the demand for electrical conduction for the operation of the third compressor would be comparable in size to direct heating, and so it is sensible for avoidance of maintenance costs, capital costs, etc., for example, to allow a slightly higher energy consumption for direct electrical heating for the electrical WT group 584 and avoid the aforementioned disadvantages. Furthermore, the design and construction complexity in the separation columns in question is reduced because only one electrical power supply has to be ensured and also the build volume of an electrically heated heat exchanger is significantly below that of a media-conducting heat exchanger.

The advantage of dividing the distributor circuit 570 into part-circuits 580, 582 is that these part-circuits can be operated at different pressure and temperature levels and hence the consumption of electrical energy for the compressors, especially the compressors downstream of the first compressor, can be reduced, and their build size can be reduced in parallel. Finally, direct electric heating means that a compressor can be dispensed with completely, especially the one with the lowest efficiency, in view of the rise in temperature in the medium per kW of electrical power for the compressor. In this embodiment, the LP and MP distributor circuits 580, 582 achieve minimization of the power costs for the operation of the compressors since the temperature levels of the circuits are adapted to the required temperature of the heat sinks and hence unnecessary compression stages are avoided and/or a respective reduction in the vapour volume flow rate to be compressed is achieved.

The embodiment as shown in FIG. 5 shows an embodiment analogous to FIG. 2, wherein the distributor circuit 570 is of greatly simplified design; the distributor circuit 570 incorporates just one heat exchanger, here the heat exchanger 302 from the bottoms circulation system of the separation tank 300 with a very high or the highest energy requirement for the plant 100. It was also possible to simplify the distributor circuit 570 because of the incorporation of just the one heat exchanger 302 with respect to the compressors in the first conduit branch 571, in that only one compressor 192 was incorporated.

In principle, the distributor circuit 570 incorporating the heat exchanger 302 could also incorporate the heat exchangers 206 of the reaction unit 102 and/or the heat exchanger 158 of the conditioning unit 104.

The example shown in FIG. 5 shows the option in which the heat exchanger 304 (condenser) as heat source is directly connected to the heat exchanger 206, the feed to the postreactor 210 (not shown), and heat exchanger 158 incorporated into the (feed) conduit 153 upstream of the main reactor 200, as heat sinks. This option of direct interconnection of individual heat exchangers is shown bottom left in FIG. 5. For better understanding, the heat exchange 206 and the conduit 116 are thus shown twice in this FIG. 5. This selective direct interconnection and distribution of the heat from the condenser 304, compared to the incorporation of the heat sinks in the distributor circuit 570 according to FIG. 2 or FIG. 3, as described above, results in an increase in efficiency of 30% to 40%, especially when at least one energy coupling is an integrated material-based EC, in that heat exchanger 304 is coupled to the (forward) heat exchanger 158 and/or the (feed) heat exchanger 327 (not shown; similarly to FIG. 6) upstream of the first separation column 320 of the first separation stage 106B.

The embodiment, in which only the heat exchanger 302 is provided in the distributor circuit 570, constitutes a particularly simple and efficient transfer system, since only one compressor stage or compressor is required, in order to cover a significant portion of the heat requirement with high efficiency. This integration alone can increase efficiency by about 40% compared to direct heating of the heat exchangers. The direct interconnection of the heat exchangers 304 as heat source with the heat exchangers 206, 158 as (consumers) heat sinks leads to an increase in efficiency of about 28%, i.e. energy saving of 28%. Thus, it is possible by both measures, the distributor circuit 570 and the direct interconnection of the condenser 304 with the heat exchangers 206, 158, to achieve an increase in efficiency of about 70%.

FIG. 6 shows a further optional embodiment. In this case, a (forward) heat exchanger 327 is provided upstream to the first separation column 320 to reduce the burden on and increase the efficiency of the interconnected closed media circuits 550, 560, 570. Advantageously, the outflowing media stream from the (forward) heat exchanger 206 of the postreactor 210, which is at a temperature level of about 110 to 125° C., is supplied as heating medium to this (forward) heat exchanger 327. In this way, the energy requirement of the heat exchanger 322 in the bottoms circulation system of the first separation column 320 and hence that of the distributor circuit 570 is lowered. A particular advantage is achieved when an integrated material-based EC of the heat exchanger 304 is undertaken with the (feed) heat exchanger 327 downstream of the pressure control unit 222 and upstream of the first separation column 320 of the first separation stage 106B (not shown).

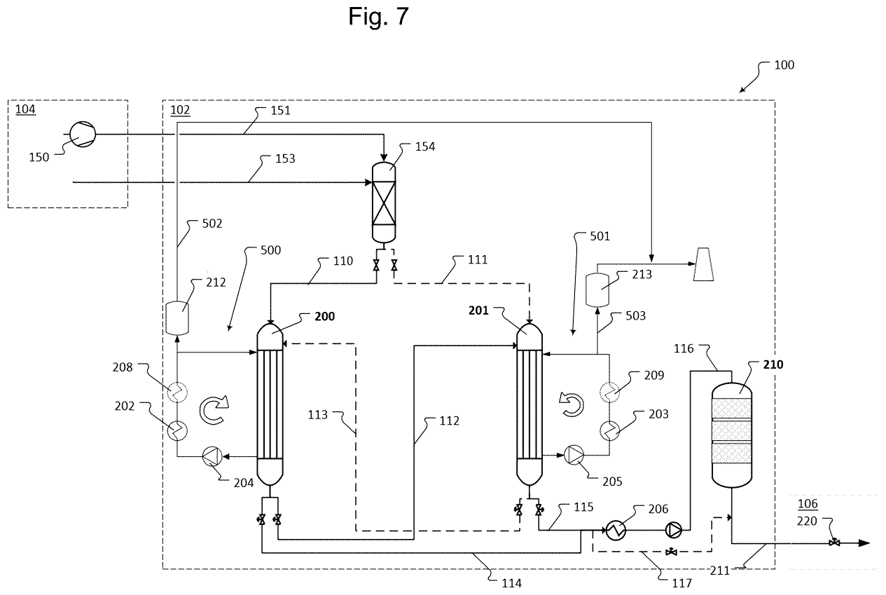

FIG. 7 shows an improved variant of the reactor unit 102. Two main reactors 200, 201 here are switchably connected to one another in series. Some of the valves/valve units that can be controlled by open- and/or closed-loop control are shown in FIG. 5; others can be provide if required by the person skilled in the art in order to ensure safe operation of the two main reactors 200, 201. The two main reactors 200, 201 are each incorporated into a cooling circuit 500, 501, with which the fixed bed reactor 200, 201 is kept in each case at a permissible, cooled reaction temperature. In the interconnection of the main reactors 200, 201 illustrated and shown with solid lines, the flow passes first through the first main reactor 200 via the conduit 110 coming from the mixing vessel 154 and the first part of the reaction takes place in this main reactor 200.

Downstream via a lower outlet and the conduit 112, the mixture of matter is fed into the top of the second main reactor 201 and leaves it via a bottoms outlet and conduit 115 into the common conduit 116 as a feed into the common postreactor 210. The common conduit 116 incorporates a heat exchanger 206, by means of which the temperature level required for the postreactor 210 can be ensured. The sequence of the flow through the two main reactors can also be reversed, from the main reactor 201 to the main reactor 200. For this purpose, in an analogous manner, the flow to the main reactor 201 first passes through the conduit 111, with the conduit 110 to the top of the main reactor 200 closed. The mixture of matter leaves this fixed bed reactor via a bottoms outlet and the (intermediate) conduit 113 which is connected to the top of the main reactor 200, with the conduit 115 closed. Finally, the mixture of matter leaves the main reactor 200 via a bottoms outlet and conduit 114, which opens analogously into the common conduit 116 and thus leads to the postreactor 210. Each of the two cooling circuits 500, 501 has a conduit 502, 503 which branches off and is guided in each case through a container 212, 213, which serve as pressure equalization vessels, and further to a chimney and/or a complete oxidation unit. The conduits and valves/valve units are present and designed in such a way that each of the main reactors 200, 201 can be operated alone and the stream of matter can completely bypass the respectively other main reactor. The flow direction of the two cooling circuits 500, 501 is symbolized by an arrow, where the two cooling circuits 500, 501 are advantageously identical or substantially identical, since the two main reactors 200, 201 are operated alternately with respect to the flow direction of the reactant stream or the stream of matter as the first or second main reactor.

However, the cooling circuits 500, 501 are designed and controllable in such a way that, depending on the process-related requirements, such as, in particular, product management and/or product quality, autonomous cooling outputs or cooling functions are achieved in each case. This relates in particular to the volume flow per unit time of cooling medium and/or the temperature level or the permissible heating of the respective cooling medium.

FIG. 7 also shows an optional conduit 117 as a dashed line (bypass 2), by which the postreactor 210 can be bypassed if, for example, it has to be maintained and/or the catalyst charge has to be renewed. In this case, the temperature at least of the second main reactor in flow direction of the stream of matter is controlled such that complete reaction is ensured with the desired product quality, in particular the desired proportion of the respective isomers. The conduit branch of the conduit 117 may be provided upstream or downstream of heat exchanger 206 in flow direction, advantageously upstream of heat exchanger 206, in order also to be able to bypass it if necessary, and to be able to undertake necessary maintenance operations while the plant is running.

The main reactor 200 can be bypassed in operation of the main reactor 201 shown on the right in the picture via the conduits 111, 115 and 116. The main reactor 201 can be bypassed in operation of the main reactor 200 shown on the left in the picture via the conduits 110, 114 and 116, such that there is no flow through the (cross-) conduits 112, 113 between the two main reactors 200, 201 in each case.

The cooling circuits 500, 501 also (optionally) incorporate heat exchangers 208, 209 in the plant variant shown. These are operated with a heating medium, especially steam, and serve for (pre) heating the reaction temperature of the respective main reactors 200, 201 in the startup step of the main reactors 200. In the plant and process example shown, as already set out for FIG. 1, with a view to the aim of achieving a minimum trans/trans isomer ratio of about 17% to 23% by weight, it is advantageous when the newly catalyst-filled main reactor 200, 201 is preheated to a temperature of about 90° C. by means of the respective heat exchanger 208, 209, or the respective newly filled main reactor 200, 201 is correspondingly preheated.

The preheating to about 85 to 95° C. makes it possible for the reaction in the case of the present catalyst to be able to start immediately, without or substantially without recycling streams of the mixture of matter from the process until the desired reaction temperature has been attained.

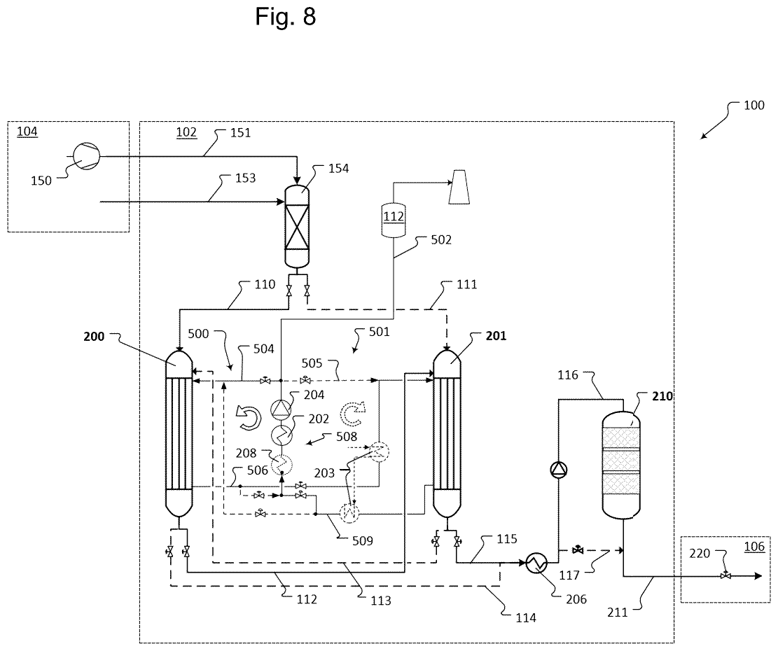

The variant of the switchable main reactors 200, 201 as shown in FIG. 8 differs from that according to FIG. 7 in that the cooling circuit 500 of the first main reactor 200 is likewise connected in series with the cooling circuit of the second main reactor 201. In this case, only one (cooling) heat exchanger 202 and only one pump 204 is provided for the common cooling and the media circuit, and so the flow through the common (central) conduit branch 508 in both cooling circuits 500, 501 is generally constant and only in one direction, irrespective of the interconnection of the two main reactors 200, 201. Of course, the “central” conduit branch need not in fact be disposed between the two main reactors 200, 201. The variant is shown as solid lines, in which the first main reactor 200 (on the left) is first fed with the reactant mixture via the conduit 110 and also the introduction (of cooling media) downstream of the heat exchanger 202 and the pump 204 is first effected via this main reactor 200. The conduits that do not carry media in this circuit or conduits that carry the mixture of matter are shown by dashed lines. In these variants too, it is possible to completely bypass the respectively other main reactor with the mixture of matter and/or the (cooling) medium. For example, in the process of filling one of the two main reactors 200, 201, the respectively other main reactor can thus continue to be operated at maximum output. The bypass of the stream of matter or of the respective main reactor 200, 201 is analogous to FIG. 7.

In the example circuit shown, the flow passes through the central conduit branch 508 and the heat exchanger 202 and is directed via the conduit 504 in cocurrent into the media space of first main reactor 200, blocking conduit 505 that leads to the common conduit node coming from the second main reactor 201. The medium leaves the first main reactor 202 via the conduit 506 at a low-level outlet and is directed to a high-level inlet into the media space of the second reactor 201 (cross-conduit). The medium likewise flows through the media space of the second main reactor 201 in cocurrent and leaves it at a low-level outlet via the conduit 509, from which a branch again flows into the central conduit branch 508, and so flow through the circuit can continue. Similarly, the flow passes firstly via the conduit 505 through the main reactor 201 shown on the right when the conduit 504 is blocked. The medium then leaves the media space of the second main reactor 201 via the conduit 509 and is directed into the media space of the other main reactor 200 at a high-level inlet. The outlet of the media space at a low-level point leads into the conduit 506 and thence via a branch into the central conduit branch 508.

The particular advantage of the switchable main reactor 200, 201 is the possibility of operating the main reactor, through which the flow passes first, at a higher temperature because the catalyst is already partly exhausted and inactivated, essentially without adversely affecting (i.e. increasing) the trans/trans isomer ratio. At the same time, more significantly heated cooling medium is obtained in the respective (circulation) heat exchanger 202, 203 of the main reactor 200, 201 through which the flow passes first. Because of the higher temperature, this hotter heat exchange medium can be better used in the plant for integrated media-based EC and/or for customary heat exchange with a reactant stream of matter.

FIG. 8, analogously to FIG. 7, shows the optional conduit 117 as a dashed line (bypass 2), which allows the postreactor 210 to be bypassed, where the conduit 117 branches off downstream of the (forward) heat exchanger 206 of the postreactor 210.

FIG. 8 also shows a plant variant (dashed line) whereby higher safety or a higher degree of freedom of temperature management is obtained in the respective (cross-) conduit 504, 505 of the cooling circuits 500, 501 in that switchable and controllable heat exchangers 203 (feed coolers) are arranged as required. In the working example shown, the two heat exchangers 202 are connected in series, since, because of the respectively inactive (cross-) conduit, the conduit 505 in the example shown, no heating takes place at the site of installation for the heat exchanger 202. The cooling media conduit or the cooling media circuit of the heat exchangers 202 can be operated in an advantageous variant in a sidestream or via a secondary or auxiliary circuit via the pump 204.

The (forward) heat exchanger 206 is described primarily in the present context and is in some cases shown in an energy coupling in which it operates as a heat exchanger 206 primarily as a heat sink, meaning that the stream of matter conducted therein is heated. Because of the dependent mode of operation of the postreactor 210 with adaptation to the main reactor 200, 201, cooling of the stream of matter in the (feed) conduit 116 upstream of the postreactor 210 may be required at least temporarily, in particular permanently, because about 10% to 20% of the conversion occurs in the (adiabatic) postreactor, such that the stream of matter is heated up to about 140° C., measured in the outlet. Thus, independently of the embodiments and variants of the plants and of the process that are described herein, it is possible to provide an adapted energy coupling for cooling (heat exchanger 206 operates as a heat source). Alternatively or additionally, supplementary cooling may be provided via a modified or additional energy coupling.

The advantage of this sidestream or secondary/auxiliary circuit of a coolant via the heat exchangers 203 has the great advantage that this additional cooling output only needs to be called on as required and, with a low degree of complexity, a greater extent of closed-loop temperature control as required is possible in the respective second of the two series-connected main reactors.

Overall, a multitude of customary open-loop and closed-loop control elements that are known to the person skilled in the art and are necessary or advisable for an advantageous process regime are not shown, such as sensors (flow, temperature, pressure etc.), displays, setting and control elements (especially valves, further pumps, compressors), collecting vessels etc., and should be added if required. In particular, when “a” pump or “a” compressor is mentioned, this also means customary redundancies from at least two parallel aggregates, especially of two parallel pumps or two parallel compressors. In an analogous manner, “a heat exchanger” should not be understood to be limiting and, with the respective local heat exchange function, also means arrangements of heat exchangers that are connected in series or in parallel, including redundant heat exchangers, which in this context does not mean interconnections with at least one further heat exchanger and a locally different heat exchange function.

All heat exchanges and condensers are fundamentally designed such that indirect heat transfer occurs and there is no physical mixing of reactants, product, by-products and/or solvent with the (heating/cooling) medium, such as gas, steam, water, oil, brine etc., unless stated otherwise.

Even if components such as valves, pressure control unit, isolators etc. are shown individually or separately in the present context for simplified description and to some degree were not mentioned individually, this should not be read in a limiting manner; instead, the person skilled in the art is able to combine two or more of these components in a valve unit or control unit as required or provide multiway valves instead.

In the present case, what is meant by “upstream” or “downstream” is the arrangement and/or flow direction of the product-rich stream of matter, unless stated otherwise. Furthermore, “media”, “media stream”, “media conduit”, etc., always means a heating or cooling medium or the associated conduit, unless stated otherwise.

The terms “bottoms outflow”, “bottoms output”, “bottoms outlet” or “bottoms discharge” are to some degree used synonymously, as are analogously the terms “tops outflow, output, outlet or discharge”.

Furthermore, the expression “heat exchanger operated as a condenser” should be interpreted broadly, and also means incomplete condensation or cooling of the supplied stream of matter, and so a “heat exchanger operated as a condenser” is also synonymously named “condenser” in some cases.

The term “bottoms pump” means a pump incorporated into a bottoms circulation system of an apparatus (separation column, vessel, etc.) and/or a pump downstream of the bottoms outlet of an apparatus, which is intended to convey liquid stream of matter.

For all embodiments and variants of the plant and of the process, it may generally be the case that the second separation column 340 and the third separation column 350 are executed as a single column, in particular as a dividing wall column (not shown). In this case, the separation functions of the second separation column 340 and of the third separation column 350 can advantageously be performed at least partly, ideally completely, by means of the dividing wall column (not shown), as known, for example, from documents EP 012 62 88 B1 or EP 012 23 67 A2.

Overall, a large, energy benefit can be achieved with the plant according to the invention and the process, wherein a significant reduction in the externally supplied energy flows was enabled, in particular saving of large amounts of (external) heating steam.

1. A plant for hydrogenation of methylenedianiline (MDA; reactant1) with a hydrogen donor (reactant2),
comprising a conditioning unit for the reactants, a reactor unit and a separation unit, wherein
the conditioning unit comprises (feed) conduits for reactant1, reactant2 and at least one solvent, at least one heat exchanger in at least one (feed) conduit, at least one mixer for mixing the reactants and/or at least one reactant with at least one solvent;
the reactor unit comprises at least one fixed bed reactor as main reactor with an immobile catalyst packing, wherein the at least one (first) main reactor comprises:
a first flow pathway for the mixture of matter through the immobile catalyst packing and another separate closed flow pathway for a heat exchange medium outside the catalyst packing, and wherein
the media circuit incorporates a heat exchanger;
the separation unit comprises at least: a first separation stage for substantia separation of the solvent and a second separation stage for separation of the at least one reactant and/or at least one by-product from the product,
wherein
i) a collection circuit for a closed first media circuit is included, in which at least one heat source incorporated is the heat exchanger of the main reactor and/or at least one heat exchanger of the separation unit and a first evaporator,
ii) an intermediate circuit for a closed second media circuit is included, incorporating the first evaporator, at least one compressor and a second evaporator, and where
iii) at least one distributor circuit for a closed third media circuit is included, in which at least one heat exchanger of the reactor unit, at least one heat exchanger of the conditioning unit and/or at least one heat exchanger of the separation unit is heat-exchangingly incorporated as a heat sink.

2. The plant according to claim 1, wherein, in the intermediate circuit, a heat exchanger is incorporated crosswise into the incoming conduit branch and the return conduit branch, where the first conduit branch is incorporated into a first interior of the heat exchanger and the return conduit branch into the second interior of the heat exchanger.

3. The plant according to claim 1, wherein the incoming conduit branch of the intermediate circuit incorporates at least two compressors.

4. The plant according to claim 2, wherein at least one cross-conduit is provided in the intermediate circuit between the incoming and return conduit branch and branches off from the return conduit branch at a conduit node and enters the incoming conduit branch at a conduit node, where the branch is advantageously disposed between the heat exchanger and the first evaporator, and the inlet is incorporated into the incoming conduit branch either between the heat exchanger and the at least one compressor or between two compressors.

5. The plant according to claim 1, wherein the media circuit of the collection circuit incorporates a collection tank and a pump, where a heat exchanger is disposed upstream of the collection tank in the return conduit branch and/or on the suction side of the pump in the incoming conduit branch.

6. The plant according to claim 1, wherein the distributor circuit comprises a first conduit branch as a vapour-conducting feed, where the feed conduit branch incorporates at least one compressor.

7. The plant according to claim 6, wherein a (bottoms) conduit leads from the second evaporator to the pressure side of the one compressor and to the suction side of a downstream further compressor, where this (bottoms) conduit incorporates a pump and/or the (bottoms) conduit or at least one branch of the (bottoms) conduit is guided from the second evaporator into the incoming conduit branch between two compressors, and the (bottoms) conduit has at least two branches, where each branch of the (bottoms) conduit is guided between two of the compressors, and where at least one branch of the (bottoms) conduit comprises a pressure regulator.

8. The plant according to claim 1, wherein the distributor circuit comprises a further conduit branch for distribution or release of energy and a further conduit branch for media recycling, wherein the conduit branch for distribution incorporates at least one heat exchanger of the reactor unit, at least one heat exchanger of the conditioning unit and/or at least one heat exchanger of the separation unit as a heat sink.

9. The plant according to claim 8, wherein the return conduit branch of the distribution circuit incorporates a heat exchanger so as to exchange heat.

10. The plant according to claim 8, wherein at least some of the heat exchangers of the distribution circuit that are incorporated as a heat sink are connected in parallel.

11. The plant according to claim 1, wherein the heat exchangers, acting as heat source, of the collection circuit are connected in series.

12. A process for catalytic hydrogenation of methylenedianiline (MDA; reactant1) with a hydrogen donor (reactant2), the process comprises:
implementing a production by an industrial plant,
wherein
the plant is designed according to claim 1, where the main reactor is operated at a temperature in the range from 80° C. to 150° C., and where, in the intermediate circuit, by the at least one compressor, vapour compression results in at least an increase in temperature of the medium in the incoming conduit branch of 30° C. to 120° C.

13. The process according to claim 12, wherein the temperature of the reactant stream at the inlet of the main reactor is 90 to 140° C.

14. The process according to claim 12, wherein the at least one main reactor is operated at a pressure in the range from 60 bar to 120 bar.

15. The process according to claim 14, wherein the pressure in the main reactor is 70 to 110 bar.

16. The process according to claim 12, wherein it is implemented continuously and catalytically for production of methylenebis(cyclohexylamine).

17. The process according to claim 12, wherein, from time t₀, the start of the process after renewal or regeneration of the catalyst, to time t₄, the end of the process determined by renewal or regeneration of the catalyst, the operating temperature of the main reactor is increased and the temperature in the stream of matter in the inlet (feed) to the postreactor is maintained or lowered, where the increasing or lowering of the temperature is linear and/or stepwise.

18. The process according to claim 12, wherein, in the distribution circuit, the pressure in the incoming conduit branch is increased in two or more stages, with an inlet pressure of 1.5 bar to 5 bar and a temperature of 100° C. to 150° C. in the first conduit branch downstream of the second evaporator and upstream of the first compressor.

19. The process according to claim 18, wherein, in the distributor circuit, the pressure in the first conduit branch is increased in two or more stages, with a pressure of 3 bar to 30 bar and a temperature of 130° C. to 300° C. downstream of the last compressor and upstream of the first heat exchanger that acts as a heat sink.

20. The process according to claim 12, wherein the energy introduced into the first evaporator by the collection circuit by the intermediate circuit and the at least one incorporated compressor and the heat exchanger incorporated crosswise is raised by at least a factor of 1.1 to 2.5 and/or
the exit temperature of the first evaporator
is raised by at least a factor of 1.2 to 3.0.

21. The process according to claim 12, wherein the MDA (reactant1) comprises or is formed from a mixture of the following monomers: 4,4′-MDA, 2,4′-MDA and 2,2′-MDA, where the proportion of 4,4′-MDA is advantageously in the range from 75 to 98 mol %.
